(19) United States
(12) Patent Application Publication
(10) Pub. No.: US 2005/0032061 A1
(43) Pub. Date: Feb. 10, 2005

(54) Method of storing and/or delivering an oligomer and/or polymer applied on a support, and supports thereof

(75) Inventor: Yoshihide Hayashizaki, Tsukuba-shi (JP)

Correspondence Address: DARBY & DARBY P.C., P. O. BOX 5257, NEW YORK, NY, 10150-5257, US

(73) Assignees: RIKEN; KABUSHIKI KAISHA DNAFORM

(21) Appl. No.: 10/494,354

(22) PCT Filed: Nov. 5, 2002
(86) PCT No.: PCT/JP02/11492

(30) Foreign Application Priority Data
Nov. 5, 2001 (JP) 2001-339217

(51) Int. Cl. C12Q 1/68; C12M 1/34

(52) U.S. Cl. 435/6; 435/287.2; 427/2.11

(57) ABSTRACT

A method for storing and/or delivering an oligomer and/or polymer applied on at least one support, comprising the steps of: (a) applying at least one oligomer and/or polymer on at least one support; (b) folding or rolling the support; and (c) storing and/or delivering the support of step (b) is provided. Also, a support having at least one oligomer and/or polymer applied thereon, which is in the form of a folded or rolled sheet, a loose-leaf sheet or a card and a water-soluble support having at least one oligomer and/or polymer applied thereon are provided. According to the present invention, molecular substances (oligomers and/or polymers) can be stored and delivered in a simple system. This system can preserve the substances without contamination and degradation.

Methods of storing and/or delivering an oligomer and/or polymer applied on a support, and supports thereof.

### TECHNICAL FIELD

The present invention relates to Methods of storing and/or delivering an oligomer and/or polymer applied on a support, and supports thereof.

### BACKGROUND ART

Biological samples and material have been traditionally stored and distributed in the form of heat-sealed glass vials. This system has the disadvantage of keeping labels attached and maintaining records of the sample characteristics as well as the need to provide as many vials as the samples or material that are desired to be stored or delivered.

An alternative system for storing and/or delivering a molecular substance fragment of interest is in the form of a booklet formed by laminating a plurality of sheet-like supports on which a high molecular fragment, for instance DNA, RNA or PNA, is fixed or printed (U.S. Pat. No. 6,258,542). However, this delivery system requires the manufacturing of the support in the form of a booklet.

A further problem in the art related to the delivery of support comprising a molecular substance is the contamination and deterioration of the molecular substance of interest desired to be stored or delivered. During preparation, delivery and reception of the support comprising the molecular substance, several operators handle the support and can potentially contaminate it. Also environmental elements (for instance, humidity, UV rays, and the like) that are encountered during storage and delivery can severely compromise the quality of the molecular substance applied on the support. Contamination is a serious problem, which may compromise the use of the molecular substances when they are recovered from the support.

For instance, in the case of using DNA recovered from a support for PCR purposes, contamination may severely compromise the PCR quality.

U.S. Pat. No. 5,092,466 discloses a system for storing biological samples which comprises the steps of preserving the biological samples in hermetically sealed packets and mounting them on a film. The sealed packet is then removed from the film by punching out a portion of the film on which the packaged sample is mounted.

However, this system requires sealing systems and the preparation of the hermetically sealed packet and film. Further, the biological sample needs to be extracted from the film and the sealed package before use and has an inevitable risk for contaminating and deteriorating the biological sample.

Therefore, in this field of art, there is a need for a simple storage and delivery system for molecular substance of interest which can preserve the substance without causing contamination and degradation.

The embodiments of the present invention solve the problems in the art by providing supports which are easy to prepare, store and deliver, and by reducing or eliminating the risk of contamination and degradation.

### DISCLOSURE OF INVENTION

The present invention solves the problem in the art by providing a storage and/or delivery system in a simple form.

The invention also provides a method for preparing a simple system for storage and/or delivery.

The invention further provides a method for storing and/or delivering molecular substances of interest.

The inventor has prepared various forms of support having at least one oligomer and/or polymer applied thereon.

The subject matters of the invention are as follows:

1. A method for storing and/or delivering an oligomer and/or polymer applied on at least one support, comprising the steps of:
(a) applying at least one oligomer and/or polymer on at least one support;
(b) folding or rolling the support; and
(c) storing and/or delivering the support of step (b).

2. The method of item 1, wherein in step (b) the support is folded one or more times.

3. The method of item 1, wherein in step (b) the support is rolled.

4. The method of item 2 or 3, wherein the support folded or rolled is then inserted into a container.

5. A method for storing and/or delivering an oligomer and/or polymer applied on at least one support, comprising the steps of:
(a) applying at least one oligomer and/or polymer on at least one support;
(b) inserting the support in a container; and
(c) storing and/or delivering the support of step (b)

6. The method of item 4 or 5, where in the container is an envelope, a bag, a can, a box, a jar, an ampulle or a tube.

7. The method of item 4 or 5, wherein the support is a loose-leaf sheet.

8. The method of any of the previous items, wherein the support is made of a water-soluble, water-dissolvable and/or water-insoluble material.

9. The method of any of the previous items, wherein the support is in the form of a sheet.

10. The method of item 9, wherein the support is in the form of a loose-leaf sheet.

11. The method of item 8, wherein the water-insoluble material comprises cellulose as a major component.

12. The method of any of the previous items, wherein the support is covered with chitin and/or chitosan.

13. The method of any of the previous items, wherein in the step (a), the oligomer and/or polymer is applied in the form of a solution comprising chitin and/or chitosan as a binding component.

14. The method of item 8, wherein the support is a wafer.

15. The method of any of the previous items, wherein the support is in the form of a card.

16. The method of item 15, wherein a plurality of supports are in the form of a card and they are inserted into a container and stored and/or delivered.

17. The method of item 16, wherein the container is a box.

18. The method of any of the previous items, wherein the support is paper.

19. The method of any of the previous items, wherein the support is covered with a matrix for preserving the oligomer and/or polymer without contamination and/or degradation.

20. The method of any of the previous items wherein a means for preserving the oligomer and/or polymer without contamination and/or degradation is applied on the support or applied between two supports.

21. The method of item 20, wherein the means for preserving without contamination and/or degradation is a sheet-like means.

22. The method of any of the previous items, wherein at least two kinds of oligomer and/or polymer are applied on the support or supports.

23. The method of any of the previous items, wherein the oligomer is selected from the group consisting of oligonucleotide, oligopeptide, oligosaccharide, PNA and a mixture thereof.

24. The method of any of the previous items, wherein the polymer is selected from the group consisting of polynucletide, polypeptide, polysaccharide, PNA and a mixture thereof.

25. The method of any of the previous items, wherein the oligomer and/or the polymer is a fragment or a complete molecule.

26. The method of item 23 or 24, wherein the oligonucleotide or polynucleotide is selected from the group consisting of genomic DNA, cDNA, RNA, mRNA, PNA and a combination thereof.

27. The method of item 23 or 24, wherein the oligonucleotide or polynucleotide is selected from the group consisting of a fragment, an EST sequence, a long strand, a full-coding and a full-length sequence.

28. The method of any one of items 23-27, wherein the oligonucleotide and/or polynucleotide is a PCR primer or an oligonucleotide probe.

29. The method of any of the previous items, wherein the oligomer and/or polymer is applied on the support by fixing or printing it on the support.

30. The method of item 29, wherein the oligomer and/or polymer is applied on the support by applying the solution of the oligomer and/or polymer directly to the support by a pin, syringe or ink-jet printer.

31. The method of any of the previous items, further comprising the step of performing PCR in-situ or treating in-situ the oligonucleotide and/or polynucleotide on the support with an appropriate restriction endonuclease.

32. The method of any of the previous items, further comprising the step of recovering the oligomer and/or polymer by elution from the support.

33. The method of any of the previous items, wherein the support comprises a bar-code, a chip or a label comprising information regarding the position of the oligomer and/or polymer on the support, and the oligomer and/or polymer identification code or number.

34. The method of item 32, wherein the recovering is carried out by inserting the support in a device and performing the elution and recovery from the support automatically by the device.

35. The method of item 34, wherein an operator selects the oligomer and/or polymer of interest and the device automatically elutes and recovers the oligomer and/or polymer of interest from the support.

36. A support having at least one oligomer and/or polymer applied thereon, which is in the form of a folded or rolled sheet, a loose-leaf sheet or a card.

37. A water-soluble support having at least one oligomer and/or polymer applied thereon.

38. The support of item 37, which is a wafer.

39. The support of any one of items 36-38, wherein at least two kinds of oligomer and/or polymer are applied on the support or supports.

40. The support of anyone of items 36-39, wherein the oligomer is selected from the group consisting of oligonucleotide, oligopeptide, oligosaccharide, PNA and a mixture thereof.

41. The support of anyone of items 36-40, wherein the polymer is selected from the group consisting of polynucletide, polypeptide, polysaccharide, PNA and a mixture thereof.

42. The support of anyone of items 36-41, wherein the oligomer and/or the polymer is a fragment or a complete molecule.

43. The support of any one of items 36-42, wherein the oligonucleotide or polynucleotide is selected from the group consisting of genomic DNA, cDNA, RNA, mRNA, PNA and a combination thereof.

44. The support of any one of items 36-43, wherein the oligonucleotide or polynucleotide is selected from the group consisting of a fragment, an EST sequence, a long strand, a full-coding and a full-length sequence.

45. The support of any one of items 36-44, wherein the oligonucleotide and/or polynucleotide is a PCR primer or an oligonucleotide probe.

46. The support of any one of items 36-45, wherein the oligomer and/or polymer is applied on the support by fixing or printing it on the support.

47. The support of item 46, wherein the oligomer and/or polymer is applied on the support by applying the solution of the oligomer and/or polymer directly to the support by a pin, syringe or ink-jet printer.

48. The support of any one of items 36-47, which is inserted into a container.

49. The support of item 48, wherein the container is an envelope, a bag, a can, a box, a jar, an ampulle or a tube.

50. A method for preparing the water-soluble support of any one of items 36-49, comprising the step of applying at least one oligomer and/or polymer on at least one water-soluble support.

51. A method for storing and/or delivering the water-soluble support of any one of items 37-49, comprising the steps of:
(a) applying at least one oligomer and/or polymer on at least one water-soluble support; and
(b) storing and/or delivering the support of step (a)
52. A method for removing an oligomer and/or polymer from the water-soluble support of any one of items 37-49, comprising the steps of:
(a) dissolving the water-soluble in an aqueous solution; and
(b) separating the oligomer and/or polymer from the aqueous solution of step (a).

53. The method of claim 51, wherein the oligomer and/or polymer is nucleic acid and the method is carried out in presence of an enzyme requiring Mg++, further comprising a step of adding excess of Mg++.

54. The method of claim 54, wherein the enzyme requiring Mg++is a polymerase.

55. The method of claims 53-54, wherein the step of addition of excess of Mg++ is an amplification step.

The term “oligomer” for the purpose of the present description is defined as any substance or type of substance that is composed of molecules comprising a small number of constitutional units; the units may be of one or more species. The term “polymer” for the purpose of the present description is defined as any substance which is composed of molecules comprising a large number of constitutional units (or “mers”) and may be one or more species.

Examples of the oligomers according to the invention are oligonucleotides, oligopeptides, oligosaccharides, PNAs or mixtures thereof. Examples of the polymers according to the invention are polynucleotides, polypeptides, polysaccharides, PNAs and mixtures thereof. According to the invention, therefore, the oligomer and/or polymer to be applied or printed on the support and/or printed material can be, for example, DNA, cDNA, RNA, PNA, plasmids, primers, proteins, enzymes, an the like. The support and/or printed material according to the invention is therefore be a quick, efficient and inexpensive sample delivery system. For instance, cDNA clones can be advantageously delivered in this way. Further, solutions for example buffers, applied or adsorbed on the support are also within the scope of the present invention. These solutions however are not limited to buffers.

The support according to the invention can be attached, added or included to a printed material in any form, for example, as a page of the printed material, a leaflet page, a page of the printed material that can be removed or cut from the printed material at predetermined breaking lines or marked lines, a free page added to the printed material, a card attached or fixed to a page or the cover of the printed material or inserted into a pocket fixed on a page or on the cover, in form of a wafer and the like. Some not limitative examples of printed materials and supports are shown in the figures. However, the printed material and support according to the invention are not limited to those shown.

Printed material according to the invention includes any kind of publication known in the art, like journals, magazines, articles, books, volumes, booklets, leaflets, pamphlets, reports, posters, abstracts collections, cards, labels, and the like.

In the invention, a support maybe made of any material, provided that at least one oligomer and/or polymer is applied thereon. For example, the support can be made of a water-soluble, water-dissolvable or water-unsoluble material. Examples of the water-soluble materials include a wafer (according to the definition The American Heritage Dictionary, 1980)(wafer is known as “oblate” in Japanese). A water-soluble material can comprise for example wheat powder paste (Stedman's English-Japanese Medical Dictionary, 3^{rd} Edition). The wafer can be in any form (see for example The American Heritage Dictionary). The wafer can preferably be in the form of thin paper like sheet. Water-soluble material is however not limited to the wafer. Water-dissolvable material indicates material which dissolve in water in its constituting components releasing the oligomer and/or polymer in water. An example of water-dissolvable paper is the paper manufactured by Mishima Paper Co, Ltd., Japan. The Mishima paper is made using carboxymethylcellulose. A description of this kind of paper can also be found in Japan. Soc. Col. Mater. 64(11), Nov. 1991, pp. 696-701. When this paper is immersed in water, the fibers constituting the paper separate from each other and as consequence the oligomer and/or polymer is released into water. In the present invention, 60MDP Mishima paper was used.

Examples of the water-unsoluble materials include materials made of cellulose.

In the invention, a material manufactured from cellulose as a major component, more specifically, ordinary plain paper copy (PPC) paper and the like may be used as the support. Water-dissolvable paper (ex. Mishima paper) as above described can also be used. Also, FTR® paper (Whatman (UK), see U.S. Pat. No. 6,294,203) can be used as the support. FTR paper consists of plain paper covered with a mixture for inactivating pathogens and preserving DNA without degradation.

Furthermore, a material prepared by coating cellulose on another sheet to reinforce the sheet with film-like cellulose may be used as the support. In this case, it is preferred that a solution of the oligomer and/or polymer according to the invention, for instance a DNA solution, is allowed to adhere to the coated cellulose film of the support.

The surface of the support to which an oligomer and/or polymer has been fixed may be coated with plastic or the like to reinforce the support.

The thickness of the support may be, for example, 1 mm or less. With a very small thickness, for example, around 0.1 mm, the workability of the support is improved even if a number of oligomer- and/or polymer-fixed supports are stacked for distribution purposes, because they are not so bulky.

### BRIEF DESCRIPTION OF DRAWINGS

FIG. 1 shows a support in the form of a sheet which is folded only once. The sheet has spots of an oligomer and/or polymer on one side and no spots on the other side. FIG. 1(a) shows one side of the support. FIG. 1(b) shows the other side of the support. FIG. 1(c) is a perspective view of the support.

FIG. 2 shows a support in the form of a sheet which is folded two or more times (i.e., 5 times). The sheet has spots of an oligomer and a polymer on both sides. FIG. 2(a) shows one side of the support. FIG. 2(b) shows the other side of the support. FIG. 2(c) is a perspective view of the support.

FIG. 3 shows a container including a folded sheet (i.e., support) having spots of an oligomer and/or polymer. The container is a box.

FIG. 4 shows a support in the form of a rolled sheet. The sheet has spots of an oligomer and a polymer on one side.

FIG. 5 shows a container including a rolled sheet (i.e., support) having spots of an oligomer and/or polymer. The container is a tube.

FIG. 6 shows a support in the form of a loose-leaf sheet. The sheet has spots of an oligomer and a polymer on one side.

FIG. 7 shows a container including loose-leaf sheets (i.e., supports) having spots of an oligomer and/or a polymer. The container is a box. The loose-leaf sheets are bound together with binding rings and covered with a cover.

FIG. 8 shows a support in the form of a card. The card has spots of an oligomer and a polymer on one side.

FIG. 9 shows a container including cards (i.e., supports) having spots of an oligomer and/or a polymer. The container is a box.

FIG. 10 shows a support in the form of a DNA chip. The DNA chip has a barcode and one or more spots of oligomers and/or polymers.

FIG. 11 is a photograph of electrophoresis which illustrates experimental results of Example 1.

FIG. 12 is a gel showing the PCR recovery of cDNA spotted on the DNA sheet (support) in presence of different amounts of Mg++ as explained in Example 3. The presence of visible bands confirms the amplification and recovery of DNA.

FIG. 13 is a schematic example of amplification and ligation of the two exons of a gene (in this case hLH) from genomic DNA as reported in Example 4. The amplification and ligation is carried out by a set of primers comprising a pair of primers for each exon. Primer HsLH1Rt of exon 1 is also partially complementary to an extremity of exon 2. Primer HsLH2F of exon 2 is also partially complementary to an extremity of exon 1.

FIG. 14 is an example of how the set of primers for the preparation of cDNA from genomic DNA may be spotted on a support. Explanation is given in Example 4.

FIG. 15 shows a gel of the two exons and full-length cDNA of hLH prepared from genomic DNA according to the experiment carried out on Example 4. Lane 1 shows markers and their relative number of bp. Lane 2 shows the band of recovered exon 2. Lane 3 shows the band of recovered exon 1. Lane 4 shows from the bottom to the top 4 bands: exon 1, an unidentified band, exon 2 and dull-length cDNA of hLH, respectively. Lanes 5, 6 and 7 refer to the same experiments as in lanes 2, 3 and 4 but without template (genomic DNA).

### EXPLANATION OF LETTERS OR NUMERALS

1: Support
2: Spot of oligomer and/or polymer
3: Container
4: Names of species and tissue from which the oligomer and/or polymer is derived
5: Spot of polymer
6: Spot of oligomer
7: Binding ring
8: Cover
9: Bar-code
10: Name of oligomer and/or polymer
11: Name of polymer
12: Name of oligomer

### BEST MODE FOR CARRYING OUT THE INVENTION

Embodiments of the printed material and/or support worked according to the present invention will be hereinafter explained by reference to the drawings.

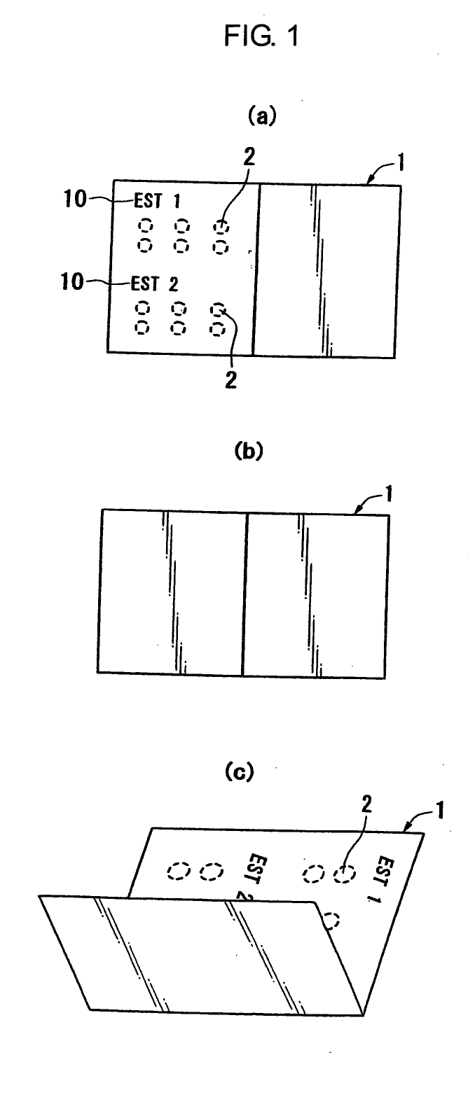

FIG. 1 shows a support (1) in the form of a sheet which is folded at least once. FIG. 1(a) shows one side of the support. FIG. 1(b) shows the other side of the support. FIG. 1(c) is a perspective view of the support. The sheet has spots (2) of an oligomer and/or polymer (e.g., DNA, cDNA, RNA, mRNA, PNA, primer, plasmid, vector, enzyme, buffer and a mixture thereof, and the like) on one side (FIG. 1(a)) and no spots on the other side (FIG. 1(b)). The name (10) of the oligomer and/or polymer is written on the support. If a scientist wants to make an experiment using the oligomer and/or polymer, he can obtain this substance immediately by cutting off a strip having the spots from the sheet and eluting the oligomer and/or polymer from the strip. When the oligomer and/or polymer is a nucleic acid, it can be advantageously recovered by amplification techniques known in the art, for example PCR by using the proper primers. These primers may be included in the same support or in a separate support of the printed material. The nucleic acid recovered can also be transferred into a prokaryotic or eukaryotic host cell, for instance E. coli, according to known techniques, for example Sambrook et al., 1989.

Applying the oligomer and/or polymer on only one half the support has the advantage that even if the support is folded, the oligomer and/or polymer will not come into contact with each other, and the possibility of molecule contamination and/or deterioration is reduced.

However, the oligomer and/or polymer can also be applied on the whole surface of the support. In that case, a means for preserving the oligomer and/or polymer without contamination and/or degradation is preferably applied on the support before it is folded.

As means for preservation, a sheet-like means can be used.

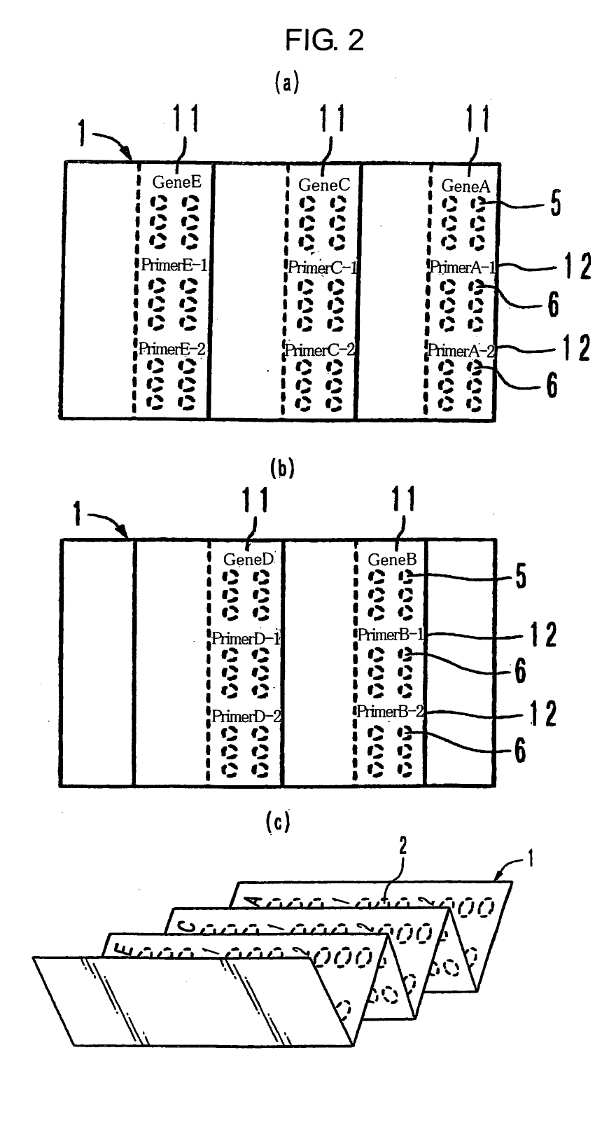

FIG. 2 shows a support (1) in the form of a sheet which is folded two or more times (i.e., 5 times). FIG. 2(a) shows one side of the support. FIG. 2(b) shows the other side of the support. FIG. 2 (c) is a perspective view of the support.

The sheet has spots (5) of a polymer (e.g., polynucleotide, polypeptide, polysaccharide, PNA and a mixture thereof) and spots (6) of an oligomer (e.g., oligonucleotide, oligopeptide, oligosaccharide, PNA and a mixture thereof) on both sides (FIGS. 2(a) and 2 (b)). The name (11) of the polymer and the name (12) of the oligomer are written on the both sides of the support (FIGS. 2(a) and 2 (b)). If a scientist wants to make an experiment using the oligomer and/or polymer, he can obtain this substance immediately by cutting off a strip having the spots from the sheet and eluting the oligomer and/or polymer from the strip.

As in the embodiment of FIG. 1, the oligomer and/or polymer are preferably applied to defined portions of the support, so that even if the support is folded, the oligomer and/or polymer will not come into contact with each other, and the possibility of molecular contamination and/or degradation is reduced.

However, the oligomer and/or polymer can also be applied on the whole surface of the support. In that case, a means for preserving the oligomer and/or polymer from contamination and/or degradation is preferably applied on the support before it is folded.

As means for preservation, a sheet-like means can be used. The embodiments of FIG. 1 and FIG. 2, have the advantage that once the oligomer and/or polymer of interest is applied on the support and the support is folded, the oligomer and/or polymer is stored in the inside of the support and it is therefore protected from the subsequent handling of the support by technicians, mailmen, and the like. These embodiments also protect the oligomer and/or polymer from external environmental elements, like humidity, UV rays, rain, and the like, because they do not directly act on the oligomer and/or polymer. The embodiments of FIGS. 1 and 2, can be conveniently stored and/or delivered by inserting the support or supports into a container, for example an envelope, a bag, a loose-leaf envelope or bag for book refill, a box, or the like.

In a modification of the embodiment of FIG. 2, the oligomer and/or polymer is applied on one side of the support.

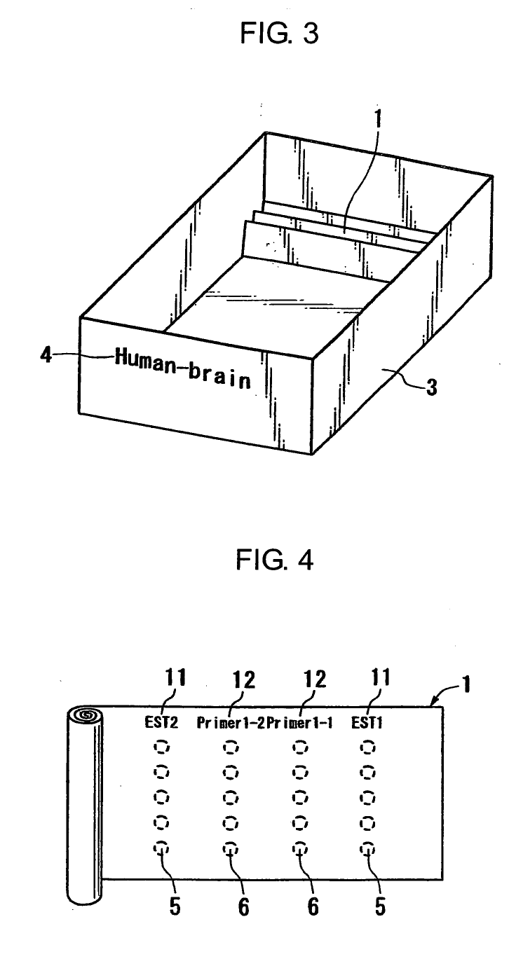

FIG. 3 shows a container (3) including a folded sheet (i.e., support (1)) prepared according to FIG. 1 and/or 2, comprising oligomer and/or polymer (e.g., oligonucleotide, oligopeptide, oligosaccharide, polynucleotide, polypeptide, polysaccharide, PNA and a mixture thereof) applied on it. The container is a box. The names (4) of species and tissue from which the oligomer and/or polymer is derived are written on the box. By containing the folded sheet in the box, oligomers and/or polymers can be pigeonholed (classified) for storage and delivery.

This system has a further advantage in that a support of oligomer and/or polymer of a particular library, or of a specific tissue are stored and/or delivered in a container separately from an oligomer and/or polymer of different library or tissue, thus avoiding the possibility of mixing up two different oligomers and/or polymers or making a mistake about the oligomer and/or polymer of interest.

FIG. 4 shows a support (1) in the form of a rolled sheet. The sheet has spots (5) of a polymer (e.g., polynucleotide, polypeptide, polysaccharide, PNA and a mixture thereof) and spots (6) of an oligomer (e.g., oligonucleotide, oligopeptide, oligosaccharide, PNA and a mixture thereof) on one side. The name (11) of the polymer and the name (12) of the oligomer are written on the support. If a scientist wants to make an experiment using the oligomer and/or polymer, he can obtain this substance immediately by cutting off a strip having the spots from the sheet and eluting the oligomer and/or polymer from the strip.

This systems has the same advantage as indicated for any of the above embodiments. A further advantage is that the oligomer and/or polymer can be applied on the whole surface of the support without contacting with each other.

Also in this system, a means for protection against contamination and/or deterioration, for example a sheet-like means, can be applied on the support before rolling it. For example, the other side of the support may be coated with an appropriate material for preserving the oligomer and/or polymer without contamination and/or deterioration.

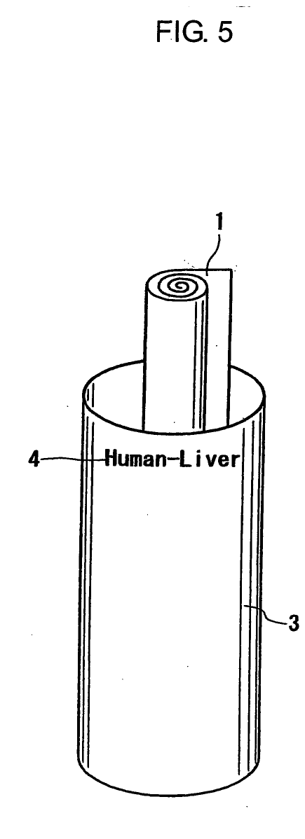

FIG. 5 shows a container (3) including a rolled sheet (i.e., support (1)) having spots of an oligomer and/or polymer (e.g., oligonucleotide, oligopeptide, oligosaccharide, polynucleotide, polypeptide, polysaccharide, PNA and a mixture thereof). The container is a tube. The rolled sheet is as shown in FIG. 4. The names (4) of species and tissue from which the oligomer and/or polymer is derived are written on the tube. By containing the rolled sheet in the tube, oligomers and/or polymers can be pigeonholed for storage and delivery.

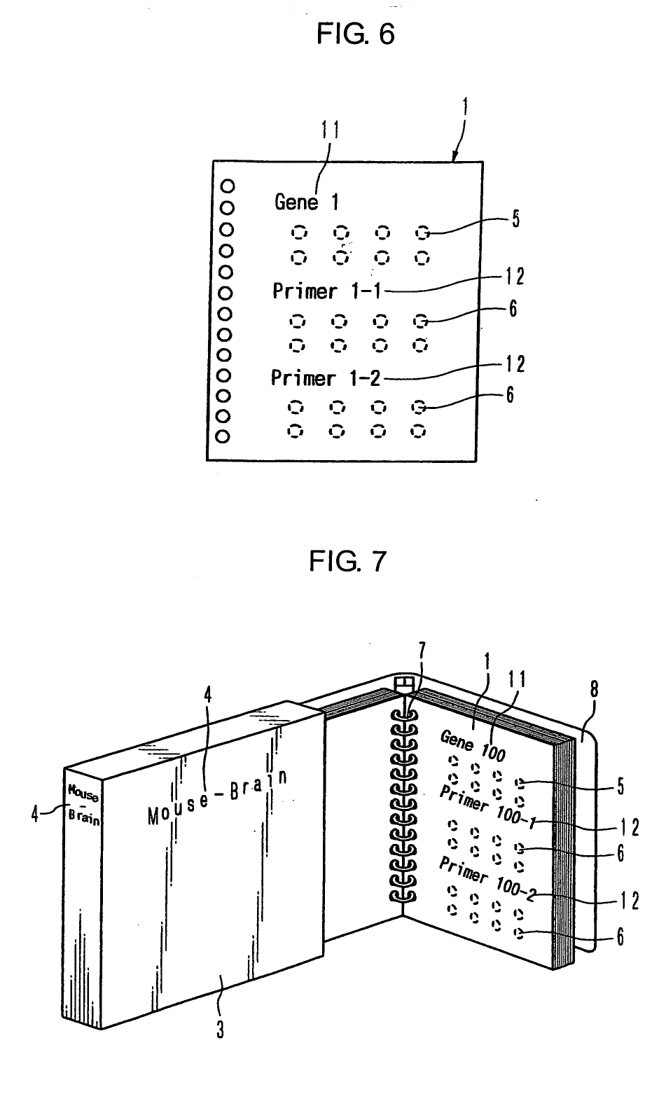

FIG. 6 shows a support (1) in the form of a loose-leaf sheet. The sheet has spots (5) of a polymer (e.g., polynucleotide, polypeptide, polysaccharide, PNA and a mixture thereof) and spots (6) of an oligomer (e.g., oligonucleotide, oligopeptide, oligosaccharide, PNA and a mixture thereof) on one side. The name (11) of the polymer and the name (12) of the oligomer are written on the support. If a scientist wants to make an experiment using the oligomer and/or polymer, he can obtain this substance immediately by cutting off a strip having the spots from the sheet and eluting the oligomer and/or polymer from the strip.

This system has a further advantage in that it can be prepared in a form ready for insertion in a book or file container. The support of this embodiment can also be processed, stored and/or delivered according to any of the approaches illustrated in FIGS. 1-5 and also FIG. 7.

FIG. 7 shows a container (3) including loose-leaf sheets (i.e., supports) having spots of an oligomers and/or polymers (e.g., oligonucleotides, oligopeptides, oligosaccharides, polynucleotides, polypeptides, polysaccharides, PNAs and mixtures thereof). The container is a box. The loose-leaf sheets are bound together with binding rings (7) and covered with a cover (8). The loose-leaf sheets are as explained in FIG. 6. The names (4) of species and tissue from which the oligomers and/or polymers are derived are written on the box. By binding the loose-leaf sheets with the binding rings and containing the sheets in the box, oligomers and/or polymers can be pigeonholed for storage and delivery.

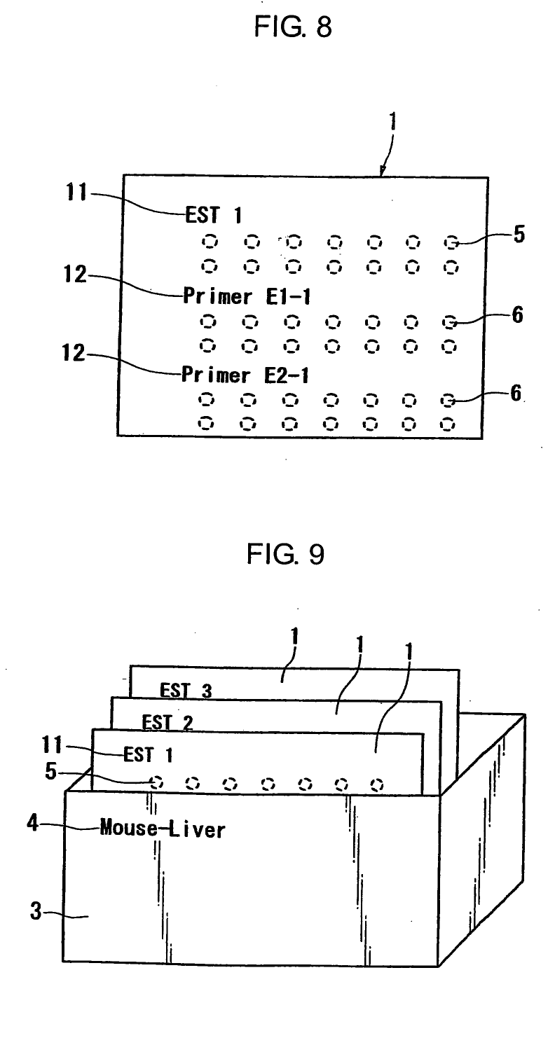

FIG. 8 shows a support (1) in the form of a card. The card has spots (5) of a polymer (e.g., polynucleotide, polypeptide, polysaccharide, PNA and a mixture thereof) and spots (6) of an oligomer (e.g., oligonucleotide, oligopeptide, oligosaccharide, PNA and a mixture thereof) on one side. The name (11) of the polymer and the name (12) of the oligomer are written on the support. If a scientist wants to make an experiment using the oligomer and/or polymer, he can obtain this substance immediately by cutting off a strip having the spots from the card and eluting the oligomer and/or polymer from the strip.

The support in the form of a card according to this embodiment can preferably comprise a bar-code, a chip or a label comprising information about the position of the oligomer and/or polymer on the card in order to distinguish the support from others. Alternatively, the position itself of the oligomer and/or polymer can represent a code and give information about the nature of the specific support. The bar code is also useful for storing information about the oligomer and/or polymer, for instance about its identification number. The use of the bar code is not limited to this embodiment but can used in any support according to the present invention.

The support in the form of a card according to the invention can also be used as it is and inserted into a device for detecting, recording, displaying, preferably on a computer screen, and printing the information about the support and the oligomer and/or polymer applied on it.

An operator after having inserted the card into the device, may preferably select the oligomer and/or polymer of interest by inputting the information into a software-guided device, for example by using a touch-screen system, and the device elutes and recovers the selected oligomer and/or polymer of interest from the support. The software, either built in or attached to the device, can also be programmed in advance and the recognition, elution and recovering of the oligomer and/or polymer selected is automatically performed by the device.

Accordingly, the invention also provides a method comprising the steps of applying at least one oligomer and/or polymer on at least one support, according to any embodiment of the invention, and recovering the oligomer and/or polymer of interest, preferably, by inserting the support in the form of a card according to the invention into a device so that the programmed device automatically recognizes the oligomer and/or polymer of interest, elutes and recovers it. Alternatively, an operator selects the oligomer and/or polymer of interest and then the device performs the elution and recovering of the oligomer and/or polymer of interest.

FIG. 9 shows a container (3) accommodating cards (i.e., supports) having spots of an oligomer and/or polymer (e.g., oligonucleotide, oligopeptide, oligosaccharide, polynucleotide, polypeptide, polysaccharide, PNA and a mixture thereof). The container is a box. The cards are as explained in FIG. 8. The names (4) of species and tissue from which the oligomers and/or polymers are derived are written on the box. By containing the cards in the box, oligomers and/or polymers can be pigeonholed for storage and delivery.

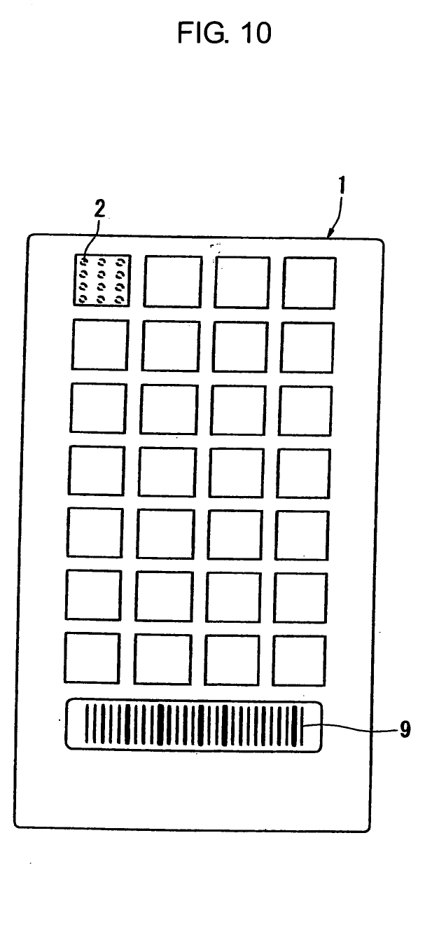

FIG. 10 shows a support (1) in the form of a DNA chip. The DNA chip has a barcode (9) and one or more spots (2) of oligomers and/or polymers. Each spot can be removed with a pin, a syringe or any other device. From the barcode, information about the oligomer and/or polymer can be obtained by using a barcode reader. Examples of the information include the position of the oligomer and/or polymer on the support, DNA sequences, amino acid sequences, three-dimensional structures of the proteins or peptides comprising the amino acid sequences, etc. The chip may be packed into a box together with other chips and a list of these chips. This type of chip is convenient in that an oligomer and/or polymer can be stored at room temperature in a smaller space. Also, it is useful in delivering oligomers and/or polymers easily.

In the aforementioned embodiments of the present invention, the supports may be covered with chitin and/or chitosan (The Merck Index, Ninth edition). Alternatively, the oligomer and/or polymer can be dissolved in a solution containing chitin and/or chitosan and then, the resulting solution is applied on the support.

A support covered with a solution or mixture comprising chitin and/or chitosan (or covered with a mixture of chitin and/or chitosan and the oligomer and/or polymer) has the advantage that the oligomer and/or polymer is applied in more stable form on the support and can be better preserved during storage and/or delivery.

The use of chitin and/or chitosan is also advantageous when it is desired to re-use the support for storage and/or delivery. The solution or mixture comprising chitin and/or chitosan may be applied on an already used support. In this case, the chitin or chitosan molecules cover the used substrate creating a new re-usable surface for further application of the oligomer and/or polymer.

In the present invention, the oligomer and/or polymer may be recovered by elution from the support. The recovering can be carried out by inserting the support in a device and performing elution and recovery from the support automatically by the device as described above for the embodiment of the support in the form of a card.

In the case of using this device, the operator may select a suitable oligomer and/or polymer.

The nucleic acid can also recovered by transferring it into a plasmid, a vector and/or an eukaryotic or prokaryotic host cell, for example E. coli or the like, according to known technique (for example Sambrook et al., 1989). The nucleic acid can be maintained and stored in this form until next use.

When the oligomer and/or polymer is nucleic acid, the present invention discloses a method for storing a nucleic acid by providing a printed material and/or a support comprising nucleic acid applied thereon, recovering the nucleic acid by transferring it into a host cell, and storing it.

The present invention therefore discloses a method for delivering biological molecules comprising the steps of applying at least one nucleic acid on the support, working the support according to any of the embodiment of the present invention, delivering the support, recovering the at least one nucleic acid by elution, amplification and/or transferring it from the support into a host molecule. In case the biological material applied on the support is a plasmid comprising cDNA, the plasmid is recovered from the support. Then the plasmid is subjected to amplification according to known technique, for instance PCR, and the cDNA is amplified. Then an electrophoresis gel is carried out (ex. Sambrook et al., 1989) and the cDNA can be recovered from the gel and used for any purpose. As said above, the amplified cDNA can also be “stored” into a host cell and kept in that form until next use. The DNA included in the host cell can also further delivered in this form.

The invention also provides a support comprising part of or all the substances necessary for an experiment, for example nucleic acid, primers, enzymes and/or solutions like buffers. All these molecules or substances can be applied on the support, worked according to the present invention, and then recovered by the reader or receiver, so that he can immediately carrying out the experiment and does not need to request the single substances, measure the concentrations of the substances and prepare them.

The invention therefore also relates to a method for providing, delivering and/or storing the oligomer and/or polymers necessary for carrying out an experiment on a support worked according to any embodiment of the present invention.

Part or all the substances necessary for carrying out an experiment, for example nucleic acid, primers, enzyme and buffers can be applied on a single support, like a card or a sheet. Accordingly, the present invention also relates to a single support comprising more than one or all the substances for carrying out an experiment. Further, the invention provides a kit for carrying out an experiment comprising more than one or all the substances for carrying out an experiment applied on a support. The more than one substance can be nucleic acid, primer(s), enzyme(s), buffer, other solutions and the like. Preferably, all the substances necessary for carrying out the experiment can be added on the support. The support can be paper, card, sheet, and the like, as described in any embodiment of the present invention. Preferably, the kit comprises more than one substance and solution for carrying out an experiment applied on a water-dissolvable paper (for instance Mishima paper) according to the invention.

Further, the present invention also provides a wafer comprising a oligomer and/or polymer and to a method for delivering and/or storing oligomer and/or polymer by applying oligomer and/or polymer on a wafer and delivering and/or storing it. The oligomer and/or polymer can be recovered by dissolving the wafer into water.

Furthermore, the present invention also provides a method for synthesizing cDNA, exons and preferably full-length cDNA, from genomic DNA. The method is carried out from the genomic DNA comprising one specific gene, for example human luteinizing hormone (hLH) (however the method is not limited to the preparation of the full-length FL or exon(s) of this gene but any gene or exon can be prepared).

The starting material is the whole genomic DNA of a cell, for instance a human cell. Genomic DNA can be purchased (for instance from BD Bioscience Clontech, US) or prepared with standard technology. A source of genomic DNA can be any biological material obtained from a patient, for example blood. For the purpose of the present invention the genomic DNA in any form, including genomic DNA prepared from blood, fluid, liquid, or any other biological material or even purchased or prepared in purified form will be here also indicated as “template” or “template DNA”.

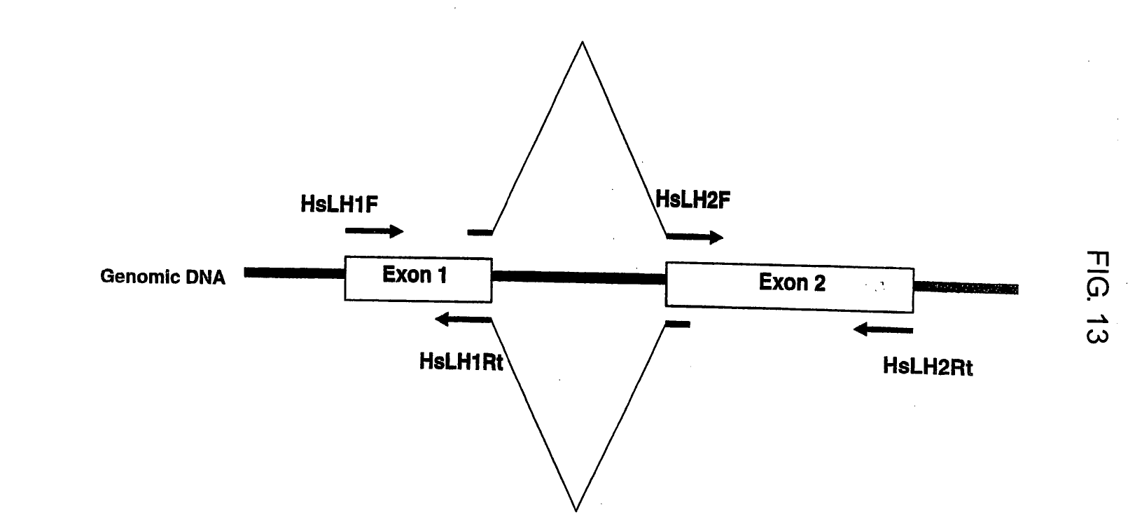

An example of the realization of this method for the preparation of FL-hLH (full-length human luteinizing hormone), which is constituted by two exons, is shown in FIG. 13 and Example 4. A set of primers capable to amplify and/or ligating the exons of the desired gene are used. The set of primers is composed of a pair of primers capable of hybridizing with each exon. One primer of a first pair of primers hybridizes with the first exon and at the same time also partially hybridize with the extremity of the other (next) exon. For instance, in FIG. 13, primer HsLH1Rt, which hybridized with exon 1, is also partially complementary to an extremity of exon 2. Primer HsLH2F, which hybridizes with exon 2, is also partially complementary to an extremity of exon 1. The final amplification products comprise a cDNA which comprises all exons of the desired gene and is therefore a FL-cDNA (full-length cDNA).

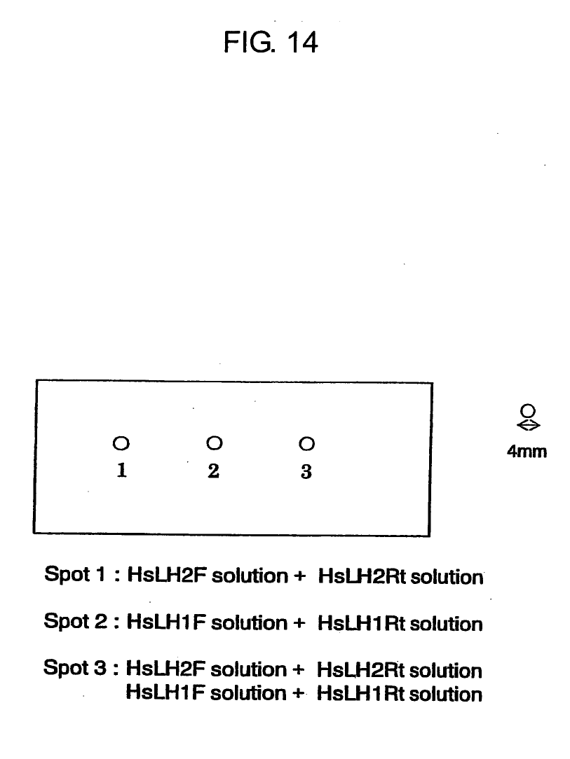

As it is shown in FIG. 13 and Example 4 a plurality of set of primers can be used of the preparatio of cDNAs from template DNA. In the example, the pair of primers for amplification of exon 1, the pair of primers for amplification and exon 2 and the set of primers (therefore comprising all the set of primers) for the amplification and ligation of the exons can be spotted in the same support. In this case, not only the full-length of the gene but also one or more exons will be synthesized (as shown in FIG. 14).

Accordingly, the present invention also provides a printed material and/or support, worked according to any embodiment of the present invention, comprising at least one set of primers applied thereon, this at least set of primers capable of synthesizing a FL-cDNA from the genomic DNA. This set of primers comprises primers for the amplification of the exons of a gene comprised in the template DNA and primers for the ligation of the amplified exons into a FL-cDNA. The support may also comprise one or more primers for amplifying one or more exons. Preferably, the support may comprise a set of primers for the synthesis of FL-cDNA and optionally further set(s) of primers for the amplification of one or more exons.

The support may further comprise one or more enzyme catalyzing these reactions (for instance Taq polymerase) and/or any solution necessary for carrying out the experiment, for instance a buffer solution.

The reader or receiver of the printed material and/or support can therefore recover the at least set of primer(s) from the support and add the template DNA, enzyme and buffer. In case the enzyme and buffer are also applied on the support, the receiver can recover all the elements from it, without need to obtain the enzyme and buffer from a different source.

The reader or receiver can then carry out the experiment, and recover the FL-cDNA obtained. The product of the experiment reaction can be applied on an electrophoresis gel, according to known technique (example, Sambrook et al., 1989) and the FL-cDNA DNA band and the bands of the exons can be identified on the gel.

Accordingly, the present invention therefore provides a method for delivering and/or storing a printed material and/or support, comprising the step of applying at least one set of primers on a support, the at least set of primers being capable of synthesizing FL-cDNA from the genomic DNA, working the support according to any embodiment of the invention, and delivering and/or storing the printed material and/or support.

A doctor, who wishes to analyse one or more particular genes of a patient may obtains a blood or other biological material sample (template) from a patient. Then, he can use the kit according to the invention comprising a support comprising at least one or more sets of primers applied thereon, each set of primers specific for amplification and ligation of a specific FL-cDNA gene. Preferably, the support further comprises the enzyme, for instance Taq Polymerase, and buffer solution. The doctor or an assistant may recover the set of primers and optionally enzyme and buffer from the support and mix them with the template DNA. Carrying out the amplification (ex. PCR) process and electrophoresis. The electrophoresis shows the FL-cDNA gene. The doctor may immediately make a diagnosis in case of deletion/insertion of the particular gene.

The FL-cDNA obtained can also used for SNP analysis (for example sequencing) or for protein expression assay.

The present invention therefore provides for a Kit and/or a diagnostic kit comprising a support comprising at least one set of primers for the synthesis of cDNA and/or FL-cDNA from a template DNA. The present invention also provides for a diagnostic method comprising the steps of 1) preparing a template DNA (blood, fluid or other biological material) from a patient, 2) recovering at least the set of primers from the support, and optionally also recovering enzyme and buffer from the support, 3) mixing the template, set of primers, enzyme and buffer, 4) carrying out amplification and/or ligation process, 5) electrophoresis and 6) determining the diagnosis on the basis of the cDNA (exon) and/or FL-cDNA obtained.

The present invention also provides a method for preparing cDNA and/or FL-cDNA from a template comprising the steps of: 1) recovering at least one set of primers and optionally enzyme and buffer from a support, 2) mixing the set of primers, enzyme and buffer with the template, 3) carrying out the amplification and/or ligation of the exons, 4) electrophoresis, 5) optionally recovering of the cDNA and/or FL-cDNA from the electrophoresis means.

Further, the above method comprises the step of analysing the obtained cDNA and/or FL-cDNA for SNP, deletion or insertion analysis or the step of expressing a peptide, polynucleotide or protein.

SNP and aberration analysis can be carried out by sequencing the cDNA and/or FL-cDNA or other known technique.

The peptide, polynucleotide or protein expression can be carried out by any peptide, polynucleotide or protein expression assay, for example the assay known as “Protein truncation test” or “Linked SP6/T7 in vitro transcription/translation kit”(2002 catalog number 188839 and 1814346, respectively), of Roche Diagnostic.

A method of preparation of full-length cDNA of hLH from genomic DNA according to the invention will be illustrated in more detail in example 4.

The present inventor has also found that the support according to the invention, like water soluble or water dissolvable paper (for instance the Mishima paper), may show chelating agent property. The result is that when an enzyme (for instance a polymerase) requiring Mg++ is used, the support binds Mg++ with the consequence of reducing the Mg++ available to the enzyme, hence the enzyme may not work efficiently. For example, when an oligomer and/or polymer (for example cDNA) is applied to a water soluble or dissolvable support, defined according to the invention, and a further step involving the use of an enzyme requiring Mg++ is carried out, the addition of excess Mg++ is preferable.

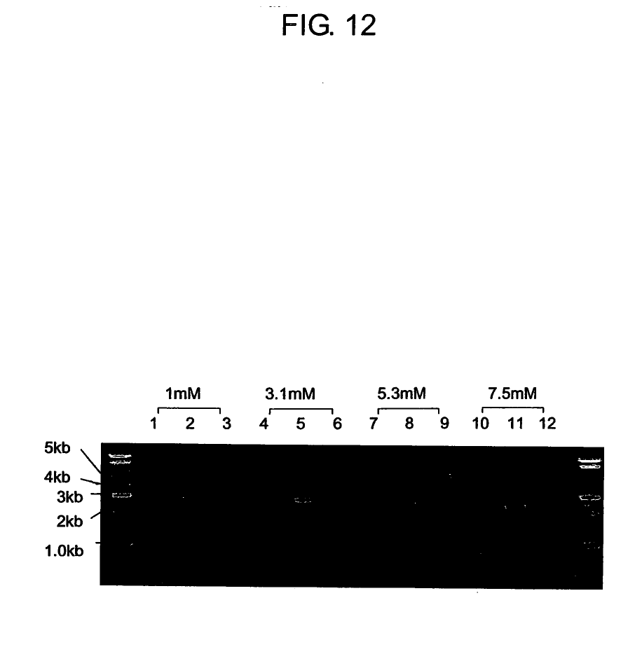

The advantage of the addition of Mg++ as above has been investigated in Example 3 and the result shown in FIG. 12. As it can be seen from the figure, at certain concentration of Mg++, for instance higher than 3.1 mM, the efficiency of amplification in improved.

The present invention therefore, provides a method comprising the step of adding excess of Mg++ in presence of an enzyme requiring Mg++ and of an oligomer and/or polymer in presence of a support.

Preferably, the invention provides a method for amplifying a nucleic acid (ex. cDNA or RNA) in presence of a polymerase and a support comprising the step of adding excess Mg++. The amount of Mg++ may be any concentration. Even low amount of excess of Mg++ may be useful. The concentration can preferably be, for instance, higher than 1 mM, higher than 3.1 mM, higher than 5.3 mM, higher than 7.5 (as shown in FIG. 12). The invention therefore, relates to any method of amplification, for instance PCR, reverse nucleic acid preparation, full-length cDNA preparation, and the like, wherein the addition of Mg++is required, for instance when an enzyme requiring Mg++ is used. The method comprising the step of adding excess (or additional) Mg++ in presence of a support is not limited to the support worked (folded or rolled) according to the invention, but can be applied to one or more support or part thereof, plurality of supports, having any shape and worked in any manner.

The support can also be inform of powder or solution preparation. Accordingly, the invention also provides a powder preparation comprising oligomer and/or polymer mixed with a carrier, for example methylcellulose. The carrier can be any inert carrier suitable to be mixed with the oligomer and/or polymer, for example any carrier usually utilized for the preparation of drugs. The powder preparation may further comprise enzyme and buffer solution.

The oligomer and/or polymer mixed with the carrier maybe nucleic acid. Further, at least one primer or set of primers can also be mixed to the preparation. Optionally, enzyme and/or buffer can also mixed to the preparation.

The invention therefore provides a method for delivering and/storing a powder preparation as above comprising mixing a carrier to an oligomer and/or polymer, and delivering and/or storing such preparation. For example, the method comprises the steps of making a preparation comprising mixing nucleic acid and at least one primer or set of primers and optionally enzyme and buffer solution with an inert carrier, and delivering and/or storing such preparation. According to a particular realization, the nucleic acid is genomic DNA, and the at least one set of primers is capable of synthesizing full-length DNA from genomic DNA as above described. The invention therefore also relates to a method for preparing full-length DNA by comprising by using the preparation as above.

The preparation can also be in solution form. Accordingly, the oligomer and/or polymer may be mixed in a liquid carrier and included in water-soluble shell. Such water-soluble shell can be for example made is the same way of shell comprising drug according to known technique. The liquid preparation may contain nucleic acid and at least one primer or set of primers, and optionally buffer solution. Alternatively, the liquid preparation may comprise primer, enzyme and buffer but no nucleic acid. The liquid preparation can be dissolved into water solution with addition with the substance (for instance enzyme) necessary for starting the reaction.

### EXAMPLES

The present invention will be explained in more detail with reference to the following examples. It should be noted, however, that the scope of the present invention is not limited by these examples.

### Example 1

First, various types of sheets each of 5 mm ×5 mm size (A) as well as 10 mm ×10 mm size (B) were prepared.

Two solutions were prepared as DNA samples. One solution (H solution) contained a plasmid DNA fragment of about 1.5 kbp including 1377 bp of λ DNA fragment inserted in pBS at a site of EcoRV at concentration of 333 ng/μl. The other solution (F solution) contained the same plasmid DNA and fountain pen ink at concentrations of 333 ng/μl and 17% (v/v), respectively.

Then, 3 μl (1 μg) each of the H solution and the F solution was spotted on a sheet of size (A), while 6 μl (2 μg) each of the solutions was spotted on a sheet of size (B), and these sheets were dried at 65° C. for 30 minutes. Thereafter, the sheet of size (A) was immersed into 200 μl of water, while the sheet of size (B) was immersed into 300 μl of water. The immersed sheets were dried at 65° C. for 10 minutes, and further they were treated at room temperature for 2 hours to conduct elution.

Phenol extraction (extracting twice with phenol:chloroform:isoamyl alcohol=25:24:1) was repeated on the eluate, and then DNA was recovered by the ethanol precipitation method.

The resulting DNA was dissolved in 10 μl of water, and PCR was conducted as follows. After the first reaction at 94° C. for 3 minutes, forty cycles of the reaction were repeated at 94° C. for 1 minute and 68° C. for 2 minutes to give a final reaction volume of 25 μl. The reaction composition was as follows: M13 (SEQ ID NO:1) primer (M3-30: 0.5 μl of 10 μM 5′-CAGTCACGACGTTGTAAAACGACGGCCAGT-3′, 0.5 μl of 10 μM RV32 (SEQ ID NO:2): 5′-GATAACAATTTCACACAGGAAACAGCTATGAC-3′), 2.5 μl of ExTaq 10×buffer solution, 2 μl of 2.5 mM dNTP, 1 unit of ExTaq, and DNA. This DNA was a DNA template of about 1.5 kbp in size containing λ DNA of 1377 bp in size together with plasmid DNA molecules located at both ends.

The base sequence of DNA (SEQ ID NO:3) was as follows:

GATAACAATTTCACACAGGAAACAGCTATGACCATGATTACGAATTCGAT
ATCGCATTTTTCACCATGCTCATCAAAGACAGTAAGATAAAACATTGTAA
CAAAGGAATAGTCATTCCAACCATCTGCTCGTAGGAATGCCTTATTTTTT
TCTACTGCAGGAATATACCCGCCTCTTTCAATAACACTAAACTCCAACAT
ATAGTAACCCTTAATTTTATTAAAATAACCGCAATTTATTTGGCGGCAAC
ACAGGATCTCTCTTTTAAGTTACTCTCTATTACATACGTTTTCCATCTAA
AAATTAGTAGTATTGAACTTAACGGGGCATCGTATTGTAGTTTTCCATAT
TTAGCTTTCTGCTTCCTTTTGGATAACCCACTGTTATTCATGTTGCATGG
TGCACTGTTTATACCAACGATATAGTCTATTAATGCATATATAGTATCGC
CGAACGATTAGCTCTTCAGGCTTCTGAAGAAGCGTTTCAAGTACTAATAA
GCCGATAGATAGCCACGGACTTCGTAGCCATTTTTCATAAGTGTTAACTT
CCGCTCCTCGCTCATAACAGACATTCACTACAGTTATGGCGGAAAGGTAT
GCATGCTGGGTGTGGGGAAGTCGTGAAAGAAAAGAAGTCAGCTGCGTCGT
TTGACATCACTGCTATCTTCTTACTGGTTATGCAGGTCGTAGTGGGTGGC
ACACAAAGCTTTGCACTGGATTGCGAGGCTTTGTGCTTCTCTGGAGTGCG
ACAGGTTTGATGACAAAAAATTAGCGCAAGAAGACAAAAATCACCTTGCG
CTAATGCTCTGTTACAGGTCACTAATACCATCTAAGTAGTTGATTCATAG
TGACTGCATATGTTGTGTTTTACAGTATTATGTAGTCTGTTTTTTATGCA
AAATCTAATTTAATATATTGATATTTATATCATTTTACGTTTCTCGTTCA
GCTTTTTTATACTAAGTTGGCATTATAAAAAAGCATTGCTTATCAATTTG
TTGCAACGAACAGGTCACTATCAGTCAAAATAAAATCATTATTTGATTTC
AATTTTGTCCCACTCCCTGCCTCTGTCATCACGATACTGTGATGCCATGG
TGTCCGACTTATGCCCGAGAAGATGTTGAGCAAACTTATCGCTTATCTGC
TTCTCATAGAGTCTTGCAGACAAACTGCGCAACTCGTGAAAGGTAGGCGG
ATCCCCTTCGAAGGAAAGACCTGATGCTTTTCGTGCGCGCATAAAATACC
TTGATACTGTGCCGGATGAAAGCGGTTCGCGACGAGTAGATGCAATTATG
GTTTCTCCGCCAAGAATCTCTTTGCATTTATCAAGTGTTTCCTTCATTGA
TATTCCGAGAGCATCAATATGCAATGCTGTTGGGATGGCAATTTTTACGC
CTGTTTTGCTTTGCTCGACATAAAGATATCAAGCTTGGCACTGGCCGTCG
TTTTACAACGTCGTGACTG

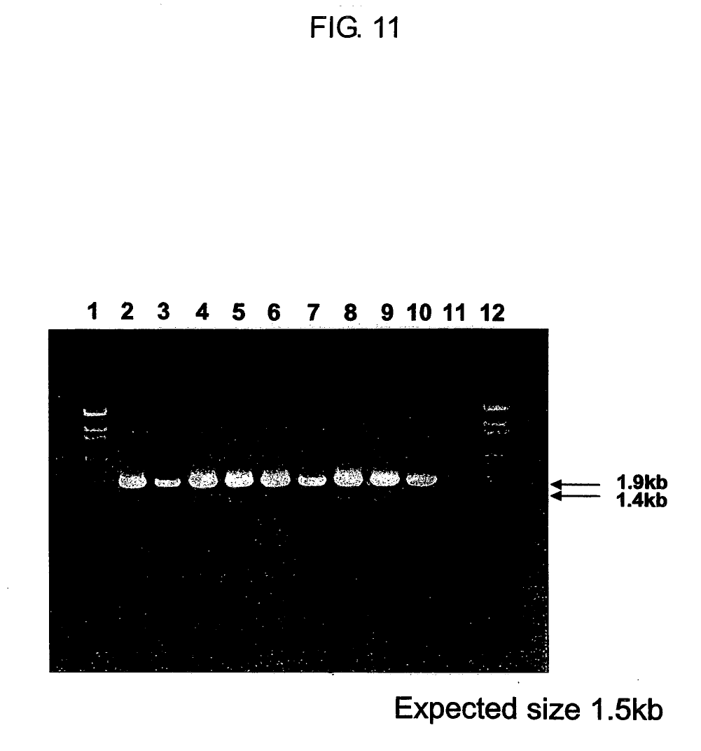

After the reaction, 5 μl of the reaction product was subjected to 1% agarose electrophoresis to detect a PCR product (about 1.5 kbp). The results are shown in FIG. 11.

Lane 1 is a DNA size marker (λ/StyI 200 μg), lane 2 shows the fragment obtained by spotting the F solution onto medical paper of size (A), lane 3 shows the fragment obtained by spotting the F solution onto medical paper of size (B), lane 4 indicates the fragment obtained by spotting the F solution onto copy paper of size (A), lane 5 shows the fragment obtained by spotting the F solution onto copy paper of size (B), lane 6 shows the fragment obtained by spotting the H solution onto medical paper of size (A), lane 7 shows the fragment obtained by spotting the H solution onto medical paper of size (A), lane 8 shows the fragment obtained by spotting the H solution onto copy paper of size (A), lane 9 shows the fragment obtained by spotting the H solution onto copy paper of size (B), lane 10 is a fragment with no sheet (positive control), lane 11 is another fragment with no sheet (negative control), and lane 12 is a DNA size marker (λ/StyI 200 μg).

Three μl of a DNA was applied to each of lanes 2, 3, and 6 while 1/50 μl of the DNA was applied to each of lanes 4, 5, 7, 8, and 9.

More specifically, the following have been found in accordance with the above described experimental results: the DNA fixed to the support in the DNA-fixed support prepared by the use of ordinary PPC or the like manufactured from cellulose as the support can be preserved at ordinary temperatures; the DNA fixed to the support can be recovered by elution from the support in the DNA-fixed support; and the DNA thus recovered by elution can be amplified by polymerase chain reaction.

In order to allow the DNA to adhere to the support, the following procedures may be adopted, namely, DNA is picked up by use of a pin, and the DNA on the pin is further transferred to the support, or a DNA solution contained in a syringe is dropped onto the support so that the DNA adheres to the support, or a DNA solution is allowed to adhere to the support in a printed state by utilizing an existing printing technique.

In this case, for example, an ink-jet printing system which is applied to ink-jet printer and the like may be utilized as the existing printing system.

In order to apply an ink-jet printing system, a DNA solution is used in place of a coloring matter such as printing ink, and the support corresponding to printing paper may be printed by the use of the DNA solution in accordance with the ink-jet printing system. Preferably, biologically and/or pharmaceutically acceptable substances can be selected for coloring the ink used for printing. Therefore, different colors can be selected and used for printing different oligomer and/or polymers so that the latter can be easily distinguished or read by the device for oligomer and/or polymer recovery, as disclosed above.

Thus, the existing printing technique can be very easily applied to a method for supporting DNA-fixation according to the present invention in that a DNA solution can be used in place of printing ink without any modification in accordance with the ink-jet printing system wherein a piezo-electric element or a heat-generating element is utilized.

In the ink-jet printing system, dots each around 20 μm to 100 μm in diameter can be usually printed, so it becomes possible to allow a DNA solution to adhere to a support in high density.

Moreover, DNA in a dried state is stable unlike other biomolecules such as protein, and it can sufficiently withstand a temperature of around 100° C. that an electronic printing or thermal transfer type printing technique including the one that is realized by laser printer can also be applied to the method for supporting DNA-fixation according to the present invention.

### Example 2

An article relating to the DNA molecule used in Example 1 was prepared which contained the title, the names of authors, abstract, introduction, materials and methods, results, discussion, acknowledgements and references. At the bottom of the article, letters “TEMPLATE” were printed using the F solution of Example 1 by an ink-jet printer (EPSON, PM-760C). As a result, clear printed letters were formed on the article.

Printing materials and supports according to the invention can be printed according to the techniques known in the art. Oligomers and/or polymers, for instance DNA solutions, may be applied, for instance spotted or printed, at defined or marked positions on the same sheet of paper or in a distinct sheet of paper or support of different size and shape. Optionally, an inert dye (for instance a red dye) can be added to the oligomer and/or polymer solution applied on the support so that the position of the spot can be visible on the support.

The sheets comprising the oligomers and/or polymers applied on them may be bound, attached or included to a book, journal, or the like and delivered to readers through bookstores and by courier. Researchers, students and readers who have any interest in the enclosed genes can recover and use them immediately in their research.

### Example 3

DNA Solution Preparation

We tested three RIKEN plasmid cDNA clones with various cDNA insert sizes (744 bp, 2440 bp and 5460 bp)(indicated as SEQ ID NOS: 4-6, respectively). The cDNAs were inserted into pBluescript according to known technique (Sambrook et al., 1989).

Plasmid DNA comprising the cDNA clones as above were purified using a Qiagen Spin Mini prep Kit (Qiagen, Japan) (alternatively, ultra-centrifugation methods, for example as described in Sambrook et al., 1989 can also be used). The plasmid DNA was dissolved in TE (10 mM Tris-HCl (pH 8.0), 1 mM EDTA. DNA concentration was adjusted to 0.1 μg/μl.

At this step an inert dye, for example a red dye, can be added to the solution in order to facilitate identification of spot position on the support at the time of recovery. However, in the present experiment the plasmid DNA solution was spot on a marked place, so that the dye was not necessary.

Preparation of DNA Sheets

About 0.1 μl of the plasmid DNA solution prepared as above was transferred onto 60MDP paper used as DNA sheet (Mishima Paper Co., Ltd, Japan) (the paper can be already printed or not) using a 96-pin tool (Multi 96-multiblot replicator VP409, Bio Medical Science Inc., US), which allowed us to spot defined amounts of DNA solution onto defined positions on the paper.

Spotted positions were easily identified by being spotted in a marked position on the paper. (Alternatively, by the presence of red dye mixed into the DNA solution as discussed in the “DNA solution preparation” as above). We spotted the plasmid DNA solution five times for each spot, for a total of about 0.5 μL of 0.05 μg of plasmids.

Extraction and Recovery of DNA

After drying the paper in air for more than 30 minutes, we extracted DNA from the sheet as follows. The piece of 60MDP paper (0.4 mm ×0.4 mm) containing the selected DNA spot was cut out from the sheet and placed into a PCR tube followed by addition of 50 g L of PCR solution. PCR solution contained 1.5 U of KOD Plus DNA Polymerase (TOYOBO, Japan), 0.2 μM of the following PCR primers: −21M13 (SEQ ID NO:7): 5′-TGTAAAACGACGGCCAGT-3′ and 1233-Rv (SEQ ID NO:8): 5′-AGCGGATAACAATTTCACACAGGA-3′), 0.2 mM each of dATP, dGTP, dCTP and dTTP and in presence of various concentrations of MgCl₂ (1 mM, 3.1 mM, 3.5 mM and 7.5 mM, respectively, as indicated in FIG. 12). After centrifuging the resulting solution, the PCR cycle was initiated. PCR cycles comprised 2 min at 94° C.; 29 cycles of denaturing (94° C., 1 min), annealing (55° C., 1 min) and extension (75 sec at 68° C.), and 15 min at 74° C. Aliquots of PCR solutions were analyzed using 1% agarose gel electrophoresis carried out according to known technique (Sambrook et al., 1989).

As an alternative, an aliquot of the solution containing the dissolved DNA sheet, can undergo PCR in a separate tube, then Escherichia coli transformation, according to known technique (Sambrook et al., 1989). Readers or receiver can keep any remaining solution as backup or for other experiments.

Result: PCR Recovery of DNAs Spotted on the DNA Sheet

FIG. 12 shows that cDNA inserts (744 bp in lanes 1, 4, 7 and 10; 2,440 bp in lanes 2, 5, 8 and 11; 5,460 bp in lanes 3, 6, 9 and 12) were amplified successfully, preferably at Mg²⁺ concentration of 5.3 mM. This test confirmed that the chosen conditions allow for an efficient spotting and extraction of DNA. In the two lanes at the extremities markers indicating the molecular weight (1, 2, 3, 4 and 5 kb) were added.

### Example 4

Method for Preparing Full-Length cDNA From Genomic DNA

Human genomic DNA used as template DNA in this example was purchased from BD Biosciences Clontech, US.

Primers for synthesizing full length cDNA of human luteinizing hormone (hLH) were purchased from Invitrogen, US.

The full-length gene of human luteinizing hormone, which is the result of the present experiment, has is 503 bp and the following sequence (also reported as SEQ ID NO:9)(herebelow the sequence corresponding to exon 1 is written in capital letters and that of exon 2 in small letters):

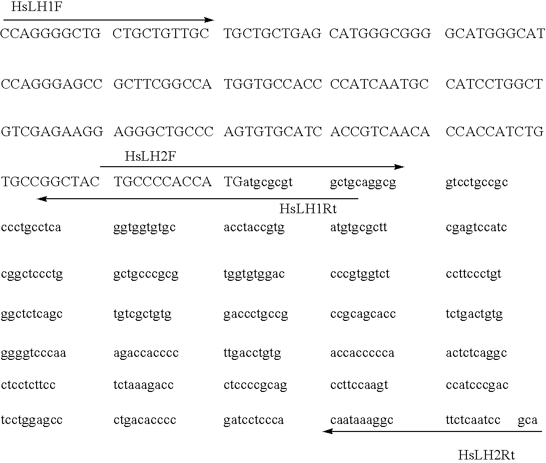

The sequence of exon 1 is reported in SEQ ID NO:10. The sequence of exon 2 is reported in SEQ ID NO:11.

The sequences of the primers (the underline indicates overlapping region) were the following:

| | |
|---|---|
| HsLH1F: | |
| CCAGGGGCTGCTGCTGTTG | (SEQ ID NO:12) |
| HsLH1Rt: | |
| cagcacgcgcatCATGGTGGGGCAGTAGCC | (SEQ ID NO:13) |
| HsLH2Ft | |
| TGCCCCACCATGatgcgcgtgctgcaggcg | (SEQ ID NO:14) |
| HsLH2R: | |
| tgcggattgagaagcctttattg | (SEQ ID NO:15) |

HsLH1F and HsLH1Rt have complementary sequences to each end of exon 1 of human luteinizing hormone, and HsLH2F and HsLH2Rt have complementary sequences to each end of exon 2 of the same gene. HsLH1Rt and HsLH2F have additional sequence complimentary to the next exon in order to ligate them to each other (FIG. 13)

The above primers were dissolved in 10 μl of TE (10 mM Tris-HCl (pH8.0), 1 mM EDTA) with the final concentration of 10 pmol/μl.

The primer solutions prepared as above was mixed for spot 1, 2, and 3.

Spot 1 solution: mixture of HsLH2F solution and HsLH2Rt solution with the ratio of 1:1;

Spot 2 solution: mixture of HsLH1F solution and HsLH1Rt solution with the ratio of 1:1;

Spot 3 solution: mixture of HsLH2F solution, HsLH2Rt solution, HsLH1F solution, and HsLH1Rt solution with the ratio of 1:1:1:1.

0.4 μl of spot 1 solution, 0.4 μl of spot 2 solution, and 0.8 μl of spot 3 solution were transferred to each corresponding spot area on a 60MDP paper (Mishima Paper Co., Ltd, Japan) as shown in FIG. 14.

After drying the paper in air for more than 30 minutes, the primers were extracted from the sheet as follows. The pieces of 60MDP paper (0.4 mm ×0.4 mm) containing the selected primer spot were cut out from the sheet and placed into three PCR tubes followed by addition of 50 μl of PCR solution. PCR solution contained 10 mM Tris-HCl, pH8.3, 50 mM KCl, 2.5 mM MgCl₂, 0.2 mM dNTP, 100 ng of template DNA (human genomic DNA, purchased from BD Biosciences Clontech, US), and 2.5 U of Taq DNA polymerase (Roche Diagnostics). PCR cycles comprised 3 min at 94° C. (50 cycles: 94° C. for 30 sec, 40 for 30 sec, 72° C. for 30 sec), and 72° C. for 1 min.

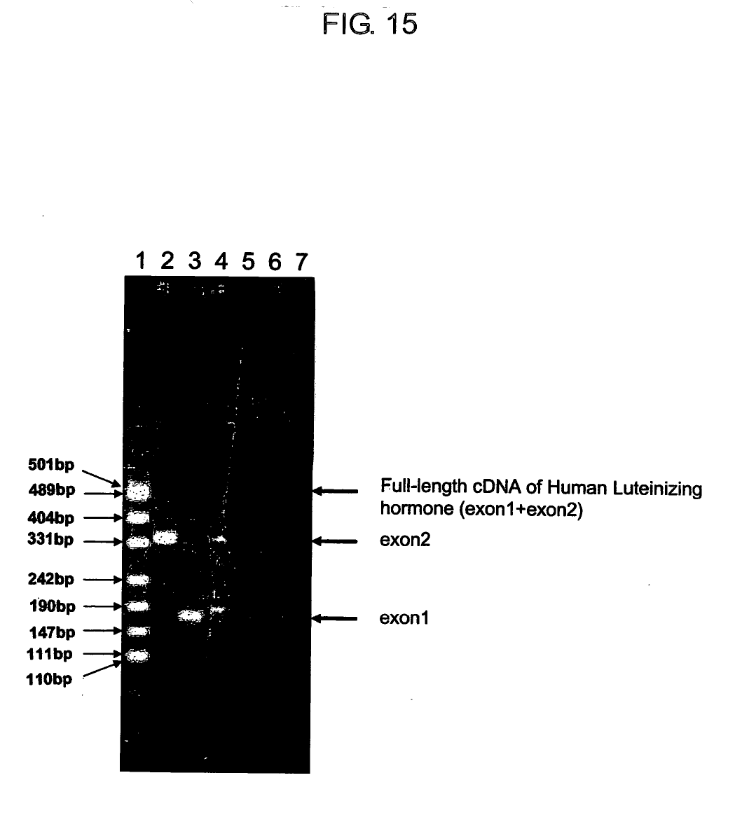

5 μl of each PCR final solution (PCR solution and primers) were analyzed using 3% NuSieve 3:1 agarose (TAKARA BIO INC., Japan) gel electrophoresis carried out according to known technique (Sambrook et al., 1989). FIG. 15 shows the result of electrophoresis. Lane 1 is pUC19/HpaII as DNA size markers (reported as base pair (bP)), and the size of each band is indicated on the left of the gel photograph (FIG. 14). Lane 2 and 3 shows that exon 2 (343 bp) and exon 1 (184 bp) were successfully amplified. Lane 4 shows that full-length cDNA of human luteinizing hormone (503 bp) was obtained. Lane 5, 6, and 7 are the control samples that contains the same substance as lane 2, 3, and 4 except the template DNA, and do not show any non-specifically amplified products.

From this result, it was proved that this technology can be used to obtain full-length cDNA from genomic DNA in one tube and by one step PCR with primers supported on said material.

### Example 5

Each of the F and H solutions of Example 1, and the DNA solution of Example 3 was spotted on ordinary PPC paper in a size of 210 mm ×297 mm and then the paper was folded once. The paper was contained in a box and stored in a shelf.

### Example 6

Each of the F and H solutions of Example 1, and the DNA solution of Example 3 was spotted on ordinary PPC paper in a size of 210 mm ×297 mm and then the paper was rolled. The paper was contained in a tube and stored in a shelf.

The entire disclosure of Japanese Patent Application No. 2001-339217 filed on Nov. 5, 2001 including specification, claims, drawings and summary is incorporated herein by reference in its entity.

All publications, patents and patent applications cited herein are incorporated herein by reference in their entity.

### INDUSTRIAL APPLICABILITY

In accordance with the present invention, a simple storage and delivery system for molecular substances of interest is provided.

This system is capable of preserving the substances without contamination and degradation.

In accordance with the present invention, a method for preparing the system is also provided.

Furthermore, in accordance with the present invention, a method for storing and/or delivering molecular substances of interest is provided.

Free Text of Sequence Listing

SEQ ID NO.1 shows the base sequence of M13 primer used in Example 1.

SEQ ID NO.2 shows the base sequence of RV32 primer used in Example 1.

SEQ ID NO.3 shows the base sequence of the template DNA used in Example 1.

SEQ ID NOS: 4-6 show the sequences of the three cDNA mouse clones tested in Examples 3.

SEQ ID NO:7 shows the sequence of −21M13 primer used for the amplification of DNA of Example 3.

SEQ ID NO:8 shows the sequence of 1233-RV primer used for the amplification of DNA of Example 3.

SEQ ID NO: 9 is the sequence of the full-length human luteinizing hormone gene (cDNA) obtained in Example 4.

SEQ ID NOS: 10-11 show the sequences of exon 1 and exon 2, respectively, of the human luteinizing hormone (hLH).

SEQ ID NOS:12-15 shows the sequences of primers HsLH1F, HsLH1Rt, HsLH2Ft, HsLH2R for the amplification of the two exons of hLH and for the synthesis of hLH full-length.

1. A method for storing and/or delivering an oligomer and/or polymer applied on at least one support, comprising the steps of:
(a) applying at least one oligomer and/or polymer on at least one support;
(b) folding or rolling the support; and
(c) storing and/or delivering the support of step (b).

2. The method of claim 1, wherein in step (b) the support is folded one or more times.

3. The method of claim 1, wherein in step (b) the support is rolled.

4. The method of claim 2, wherein the support folded or rolled is then inserted into a container.

5. A method for storing and/or delivering an oligomer and/or polymer applied on at least one support, comprising the steps of:
(d) applying at least one oligomer and/or polymer on at least one support;
(e) inserting the support in a container; and
(f) storing and/or delivering the support of step (b).

6. The method of claim 4, wherein the container is an envelope, a bag, a can, a box, a jar, an ampulle or a tube.

7. The method of claim 4, wherein the support is a loose-leaf sheet.

8. The method of the claim 1, wherein the support is made of a water-soluble, water-dissolvable and/or water-insoluble material.

9. The method of claim 1, wherein the support is in the form of a sheet.

10. The method of claim 9, wherein the support is in the form of a loose-leaf sheet.

11. The method of claim 8, wherein the water-insoluble material comprises cellulose as a major component.

12. The method of any of claim 1, wherein the support is covered with chitin and/or chitosan.

13. The method of claim 1, wherein in the step (a), the oligomer and/or polymer is applied in the form of a solution comprising chitin and/or chitosan as a binding component.

14. The method of claim 8, wherein the support is a wafer.

15. The method of claim 1, wherein the support is in the form of a card.

16. The method of claim 15, wherein a plurality of supports are in the form of a card and they are inserted into a container and stored and/or delivered.

17. The method of claim 16, wherein the container is a box.

18. The method of claim 1, wherein the support is paper.

19. The method of claim 1, wherein the support is covered with a matrix for preserving the oligomer and/or polymer without contamination and/or degradation.

20. The method of claim 1, wherein a means for preserving the oligomer and/or polymer without contamination and/or degradation is applied on the support or applied between two supports.

21. The method of claim 20, wherein the means for preserving without contamination and/or degradation is a sheet-like means.

22. The method of claim 1, wherein at least two kinds of oligomer and/or polymer are applied on the support or supports.

23. The method of claim 1, wherein the oligomer is selected from the group consisting of oligonucleotide, oligopeptide, oligosaccharide, PNA and a mixture thereof and the polymer is selected from the group consisting of polynucletide, polypeptide, polysaccharide, PNA and a mixture thereof.

24. (canceled)

25. The method of claim 1, wherein the oligomer and/or the polymer is a fragment or a complete molecule.

26. The method of claim 23, wherein the oligonucleotide or polynucleotide is selected from the group consisting of genomic DNA, cDNA, RNA, mRNA, PNA and a combination thereof.

27. The method of claim 23, wherein the oligonucleotide or polynucleotide is selected from the group consisting of a fragment, an EST sequence, a long strand, a full-coding and a full-length sequence.

28. The method of claim 23, wherein the oligonucleotide and/or polynucleotide is a PCR primer or an oligonucleotide probe.

29. The method of claim 1, wherein the oligomer and/or polymer is applied on the support by fixing or printing it on the support.

30. The method of claim 29, wherein the oligomer and/or polymer is applied on the support by applying the solution of the oligomer and/or polymer directly to the support by a pin, syringe or ink-jet printer.

31. The method of claim 23, further comprising the step of performing PCR in-situ or treating in-situ the oligonucleotide and/or polynucleotide on the support with an appropriate restriction endonuclease.

32. The method of claim 1, further comprising the step of recovering the oligomer and/or polymer by elution from the support.

33. The method of claim 1, wherein the support comprises a bar-code, a chip or a label comprising information regarding the position of the oligomer and/or polymer on the support, and the oligomer and/or polymer identification code or number.

34. The method of claim 32, wherein the recovering is carried out by inserting the support in a device and performing the elution and recovery from the support automatically by the device.

35. The method of claim 34, wherein an operator selects the oligomer and/or polymer of interest and the device automatically elutes and recovers the oligomer and/or polymer of interest from the support.

36. A support having at least one oligomer and/or polymer applied thereon, which is in the form of a folded or rolled sheet, a loose-leaf sheet or a card.

37. A water-soluble support having at least one oligomer and/or polymer applied thereon.

38. The support of claim 37, which is a wafer.

39. The support of claim 36, wherein at least two kinds of oligomer and/or polymer are applied on the support or supports.

40. The support of claim 36, wherein the oligomer is selected from the group consisting of oligonucleotide, oligopeptide, oligosaccharide, PNA and a mixture thereof and the polymer is selected from the group consisting of polynucletide, polypeptide, polysaccharide, PNA and a mixture thereof and the polymer is selected from the group consisting of polynucletide, polypeptide, polysaccharide, PNA and a mixture thereof.

41. (canceled)

42. The support of claim 36, wherein the oligomer and/or the polymer is a fragment or a complete molecule.

43. The support of claim 40, wherein the oligonucleotide or polynucleotide is selected from the group consisting of genomic DNA, cDNA, RNA, mRNA, PNA and a combination thereof.

44. The support of claim 40, wherein the oligonucleotide or polynucleotide is selected from the group consisting of a fragment, an EST sequence, a long strand, a full-coding and a full-length sequence.

45. The support of claim 40, wherein the oligonucleotide and/or polynucleotide is a PCR primer or an oligonucleotide probe.

46. The support of claim 36, wherein the oligomer and/or polymer is applied on the support by fixing or printing it on the support.

47. The support of claim 46, wherein the oligomer and/or polymer is applied on the support by applying the solution of the oligomer and/or polymer directly to the support by a pin, syringe or ink-jet printer.

48. The support of claim 36, which is inserted into a container.

49. The support of claim 48, wherein the container is an envelope, a bag, a can, a box, a jar, an ampulle or a tube.

50. A method for preparing the water-soluble support of claim 36, comprising the step of applying at least one oligomer and/or polymer on at least one water-soluble support.

51. A method for storing and/or delivering the water-soluble support of claim 37, comprising the steps of:
(g) applying at least one oligomer and/or polymer on at least one water-soluble support; and
(h) storing and/or delivering the support of step (a).

52. A method for removing an oligomer and/or polymer from the water-soluble support of claim 37, comprising the steps of:
(a) dissolving the water-soluble in an aqueous solution; and
(b) separating the oligomer and/or polymer from the aqueous solution of step (a).

53. The method of claim 51, wherein the oligomer and/or polymer is nucleic acid and the method is carried out in presence of an enzyme requiring Mg++, further comprising a step of adding excess of Mg++.

54. The method of claim 54, wherein the enzyme requiring Mg++ is a polymerase.

55. The method of claim 53, wherein the step of addition of excess of Mg++ is an amplification step.


SEQUENCE LISTING (15 sequences)

SEQ ID NO: 1 - DNA, 30 bp, Artificial Sequence
  Description of Artificial Sequence synthetic DNA
cagtcacgacgttgtaaaacgacggccagt

SEQ ID NO: 2 - DNA, 32 bp, Artificial Sequence
  Description of Artificial Sequence synthetic DNA
gataacaatttcacacaggaaacagctatgac

SEQ ID NO: 3 - DNA, 1469 bp, Lambda virus
gataacaatttcacacaggaaacagctatgaccatgattacgaattcgatatcgcatttttcaccatgctcatcaaagacagtaagataaaacattgtaacaaaggaatagtcattccaaccatctgctcgtaggaatgccttatttttttctactgcaggaatatacccgcctctttcaataacactaaactccaacatatagtaacccttaattttattaaaataaccgcaatttatttggcggcaacacaggatctctcttttaagttactctctattacatacgttttccatctaaaaattagtagtattgaacttaacggggcatcgtattgtagttttccatatttagctttctgcttccttttggataacccactgttattcatgttgcatggtgcactgtttataccaacgatatagtctattaatgcatatatagtatcgccgaacgattagctcttcaggcttctgaagaagcgtttcaagtactaataagccgatagatagccacggacttcgtagccatttttcataagtgttaacttccgctcctcgctcataacagacattcactacagttatggcggaaaggtatgcatgctgggtgtggggaagtcgtgaaagaaaagaagtcagctgcgtcgtttgacatcactgctatcttcttactggttatgcaggtcgtagtgggtggcacacaaagctttgcactggattgcgaggctttgtgcttctctggagtgcgacaggtttgatgacaaaaaattagcgcaagaagacaaaaatcaccttgcgctaatgctctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaagttggcattataaaaaagcattgcttatcaatttgttgcaacgaacaggtcactatcagtcaaaataaaatcattatttgatttcaattttgtcccactccctgcctctgtcatcacgatactgtgatgccatggtgtccgacttatgcccgagaagatgttgagcaaacttatcgcttatctgcttctcatagagtcttgcagacaaactgcgcaactcgtgaaaggtaggcggatccccttcgaaggaaagacctgatgcttttcgtgcgcgcataaaataccttgatactgtgccggatgaaagcggttcgcgacgagtagatgcaattatggtttctccgccaagaatctctttgcatttatcaagtgtttccttcattgatattccgagagcatcaatatgcaatgctgttgggatggcaatttttacgcctgttttgctttgctcgacataaagatatcaagcttggcactggccgtcgttttacaacgtcgtgactg

SEQ ID NO: 4 - DNA, 744 bp, Mus musculus
tgtaaaacgacggccagtgaattgtaatacgactcactatagggcgaattggagctccaccgcggtggcggccgcataacttcgtatagcatacattatacgaagttatggatcaggccaaatcggccgagctcgaattcgtcgacgagtggcgttgagagctcagagcttgctacagggctacagtgctccaaagcccttcaggattcactttggatacagctctttggagcctgccagcaattagaaatctttctctctcacagacaaatcatgactggaattcatcatggagaaaatgtcagccacagagaagaagcatttagcatgtttgagcacggtcatgaaatagtcatcactacatcagtgatgcacattaagcagaatgtagctgtgctcttgtggatgtgctctctcatcctggactggataatcacatggaaggcctgtgcttcactctccctagagaatgatttgcatcagtaggttataatttaggatgtgtaagaattgcctaaaacactgctggaaacttccctaaggctctggatcacacacggagggttgagaaacaactacaagccaaggacccttaaaagatgtggaagcaccggatccggccataagggcctgatccgtcgagggggggcccggtaccagcttttgttccctttagtgagggttaatttcgagcttggcgtaatcatggtcatagctgtttcctgtgtgaaattgttatccgct

SEQ ID NO: 5 - DNA, 2440 bp, Mus musculus
tgtaaaacgacggccagtgaattgtaatacgactcactatagggcgaattggagctccaccgcggtggcggccgcataacttcgtatagcatacattatacgaagttatggatcaggccaaatcggccgagctcgaattcgtcgacgctttttggcgccatggcgcaaagtcgtgttaccgatttctatgcgtgccgtcgccctggccttacgactccgcgggccaagtcgatctgtctcaccccgagccctggtggcctcgtggctcctgcgttcacccggagcagcagccgcaagcgcgcccggcccccagccgaacccgggagtgaccagcccgcgccgctcgcgcgccggaggttacggctgcctggattggactcctgccccagttctctgcctgagcccagttccccagctgagcccagccctccagctgaccctagccctccagctgaccctggctcccccgtttgcccatcccccgtcaagcgaacaaagagtacaactgtttatgttggtcaacagccgggcaagatcccctcagaggactccgtctctgagctccagtcctgcctgaggcgggcacggaagttgggagcccaggcacgggccctgagagcccgagtccaagagaatgctgtggagcctagtaccccagatgccaaggtgcccacagagcaaccatgtgtcgagaaagctcctgcctaccagcgcttccatgctctggctcagcctggtctcccaggccttgtcctaccctacaagtatcaggtgctagttgagatgttccgcagcatggacaccattgtgagcatgctccacaatcgctctgagactgtgacctttgccaaagtcaagcaaggtgttcaggagatgatgcgcaagcgctttgaagagcgcaatgtgggccagatcaaaaccgtgtatcccacgtcgtatcgcttccgccaggagtgcaatgtccccaccttcaaggacagcatcaagagatctgattaccagctcaccatcgagcccttgctgggccaggaggctggcggtgcaacccagctcacagccacgtgcctcctgcagcgccggcaagtcttccggcagaacctggtggaacgtgtcaaggaacagcacaaggtcttcctggcttcactgaacccccccatggcggtgccggacgaccagctgacccgctggcatccgcgcttcaatgtggacgaggtgcctgacattgagccagctgaactgccccagcctcctgtcacagagaagctcaccactgcccaggaagtgctggcccgtgcccggagcttgatgacacccaagatggagaaggccctgagcaacctggccctgcgctcggctgagcccggtagccctgggacctctactccaccactcccggccactccgccagccaccccacctgctgcctctccgagcgccctgaagggtgtgtcccaagcactgctagagcggataagggccaaggaggtccagaagcagctggcaaggatgacacggtgccccgagcaggagcttcgcctgcagcggttagagcgtctgccagagctggcccgcgtgctgcgcaatgtcttcgtgtctgagcggaagccggcactcactatggaggtggtctgtgcaaggatggtggacagttgccaaactgctctgagtccaggggagatggagaaacatctggtgctcctggcagagttgctgccggactggctcagcctgcatcgcatccgcacggatacctacgtcaagctggacaaggctgttgacctggctggcctcactgcgaggctggcccaccacgtccacgccgaggggctgtgactttgagctccttgcctgtttctttcatcagtacacgacgcacttacgcctttaagcctcgccagtgtgggcagctatcgttgcccatggatctcataaagtgctggcattaagttgcttcctgaggctttggggcatcccagactcagctctaggggaagtagactctgaagagtagggtatgttggatggccattgcacaaactactcaagcattaggtatggtcaagacccaagatcaaggtccaggtggaagcctaggctggcatagccttgcttccccatgagacttaagaatcacacagaccttggactttcctgatttcacgggacgctgctctgagagtgaaattgggccttctgtaaatatgtgaagtgtggtttcttttcaaaccttatatggccctgcatgtgactgctagttttggcttttaataaagtcatgtaagatttaaataaaatactactgagatggggatccggccataagggcctgatccgtcgagggggggcccggtaccagcttttgttccctttagtgagggttaatttcgagcttggcgtaatcatggtcatagctgtttcctgtgtgaaattgttatccgct

SEQ ID NO: 6 - DNA, 5460 bp, Mus musculus
tgtaaaacgacggccagtgaattgtaatacgactcactatagggcgaattggagctccaccgcggtggcggccgcataacttcgtatagcatacattatacgaagttatggatcaggccaaatcggccgagctcgaattcgtcgacgagtcgtcaggaccgcttcggctctcggagtaggaagcttggggcgctgggctggtaaggacccgcggcggggcgaagatggagtcctttaccaatgatcggcttcagcttccaaggaatatgatcgaaaacagcatgtttgaagaagagccagatgtggtagatttagccaaagaaccttgtttacatcctctggaacccgatgaagtggaatatgagccccgaggttcgaggcttctggtgcgaggtcttggtgagcatgagatggacgaggatgaagaggattatgagtcctctgcgaagctgctgggcatgtccttcatgaacagaagctcaggcctgcggaacagtgcagcaggctacaggcagagtccagatgggacttgttcattaccctctgccaggaccttagtgatctgtgtttttgtcattgtggttgcggtctctgtaatcatggtgatttatctactgcctagatgtacctttaccaaagaaggctgccacaaaacaaaccagtcagcagaactcatccagccagttgctacaaacgggaaagtgttcccatgggctcaaattaggcttcccactgccattattcctctatgctatgaacttagcctacatccaaacctaacctcaatgacattcaggggatctgtgacaatttcacttcaggctcttcaagacacacgggatatcattctccatagcacaggacataatatttcaagagtgacatttatgtcagctgtttcaagtcaagaaaaacaagttgaaattctggaatatccatatcatgaacaaatcgccgttgttgccccggaacctcttctaacaggacacaattataccctgaagatagagtattcagcaaatatatctaactcttattatgggttttatggcatcacctacacagataaaagtaatgagaaaaagtactttgcagcaactcagtttgaacctctggcagcaagatctgcttttccttgttttgatgaaccagcatttaaggccacatttatcatcaagatcacaaggaatgagcaccatactgcgttatcaaatatgcctaagaagtcatcggtccctgcagaagaaggacttattcaagatgagttttctgaaagtgtgaaaatgagcacatacctggttgctttcattgtaggggagatgaggaacctgagtcaggatgtaaatggaactctggtttctgtgtatgctgtaccagaaaaaattggtcaagttcaccatgccttggacacaactataaagcttcttgagttttatcaaacttactttgaaattcagtacccacttaagaaattggatttggtggccattcctgactttgaagcaggagcaatggaaaactggggcctgcttacattccgagaagagactcttctgtatgacaatgcaacttcttcagtagcagacagaaaactggtcactaaaatcattgctcacgaactggcacatcagtggtttggaaatctggttacaatgcagtggtggaatgacctgtggctaaatgaaggctttgccactttcatggagtatttctctgtggaaaaaatattcaaagagctcaacagttatgaagacttcttagatgctcgatttaaaaccatgaggaaagattccttgaattcgtctcatccaatatcatcatctgttcagtcttcggaacaaatagaagaaatgtttgattctctctcctattttaagggagcttctctcttgttgatgctgaaaagttaccttagtgaagatgtatttcggcatgctgtcattctttacctgcacaatcacagctatgcagctatccaaagtgatgatctctgggacagcttcaatgaggtcacagacaaaactctagatgtaaagaaaatgatgaaaacctggaccctacagaaaggattcccgttagtgacagtccagaggaaggggactgagcttcttctacaacaggaaagattttttctacgcatgcaaccagaaagtcagccttcagatacaagccacctttggcatattccaatatcctatgtcactgatggaagaaactattcagaatatcgatcagtttcactactggacaagaaatcagatgtcatcaatcttacagaacaagtgcagtgggtcaaagtcaattcaaacatgactggctattacattgttcactatgctcatgatgactggacagctctaatcaatcagttaaaaagaaacccttatgttctgagtgacaaagaccgagccaacctgatcaataacatctttgaacttgcaggtcttggcaaagtgcctcttcggatggcatttgatttgattgactatcttaaaaatgagacccacactgcaccgatcactgaagccctgttccagacaaaccttatctataatctcctagaaaaactggggcacatggacctgtcctcaagattggtggccagagtacataaattgctccagaaccaaatccagcagcagacttggacagatgaaggcacaccatccatgcgagagcttcggtcagccttgctggaatttgcctgtgcccacagcctagagaactgtaccactatggccacaaatctgtttgacagttggatggcatcaaatggaactcagagcctgcctactgatgtcatggtcactgtgttcaaggtgggagcgagaaccgagaaaggctggttgttcctctttagcatgtattcctccatgggctctgaagcagaaaagaataaaattcttgaagctcttgccagctcagaggatgtacataaactttactggttaatgaaaagtagccttgatggtgacatcatccgaacacagaagttgtcacttatcattagaacagtgggcagacattttcctggacacttgctggcttgggattttgttaaagaaaactggaataagcttgtacataagttccatctgggctcctataccatacaaagtattgttgctggatctactcacttattttcaacaaagacacatttatctgaggtccaggcattctttgaaaatcagtcagaggcaaccttgaagcttcggtgtgttcaggaggctttggaagtgattcagctgaatatccagtggatggtcaggaatctgaaaacgctctcccagtggctgtagcactcacagctgatctccggcgcccatggctctgctgctttcgcaaaggttgagtgaaggccggcctgctgctgagttgtttgcactgttaggagttctagttagctcagggcccaattgtatttttcatatcttttctgaaatgtctttaggcagtagttatttattacaaaattatattcacctgtatgtcatccatctacaataacaatgaataacttttctttgacacacaaatggagaaaaaagtcagatttgaaattttgttttatttcttagtgtccagagtattactgttacagtgcaaccaaggaaggtctgctgtaagaaccactgtgtttgtaggttgctgattgatcaactgtttgttgctttttgttaaatagatacacctgggtttggtagtgcagtggcaaagacattgaaggtacagctctagacagatgacctgattgggggttctttaaaaaaagttttgtccagtttccagttttttcctgcaaaatagccaggttctaaagactttttcatatttctgttaaattgaacagctgaacagtcaggctcaaagtttcagttttccaaatatttaacactttctgcttcatcttcatgacactatgtaggaagtgttctgaactgagttaacattaattactttactcatgaattccagtacccctaactttgtatcctggtaaggccttccaccagctttctcctgtagaggagcactccatgcttgggaggatttctcctgtagaggagcactccatgccatgcttgggaggatttctgtgagcgctggtggctttgtcattactttcacccaagaatgtaatttgtttaagttgctgtttgaaccttacaaatcataccctcttctctgtgaatcatacccatgtgtgggttttactttatgttacagagtatattagaagcatttcttaaataggatcttcaagaaattaaagctttttattttgaagttcaccataataccttaaaggaataatgagaatgtggagcaggaggaagttgaaaatatggtgggctatgaattctttttttcactgttaaaaacagtgtttttatttagttgaaaaaattacaggttttttgtttgtttgtttgtttgtttatcatggccaaactacgttaaacagagtggccaagaatcaagcagaggacaagggagggagggaacccagagcagaatgtgggcgctttcactccttctgaaggagaaggattgtacttgaactttttgagctatgcagacttattcctagattgttgtgtttctttgaatcttaggagagaagctttcttcttaataactcatgagaagtctatattttgtttgaaaacagtgtctgtgtacataaaaagggaatcactcatttctgaacactgaaaacactccacagagtcagaccatagagaggccacttgtttttattcagctgttgtaaagttagaatatgtggagttatatacaaagttatagtttattttatgagaatatttttatataataaaattttaatggctgacaagctgaaaatagcctgctttaatcctagagtttgctcccaatttcatcttttctaaacagtttttgggtttttgtttgtttgtttgttttttaatttgtgctttttttagtagaaactgttattcctgagaatcatagaatcatctttctattttagtgaaataggtaattatagatctctattccccttgtatttttggtaaatgtctgacagttgtgttgatactgaaggttagtagggttttatatttttagagtgaaggacaaatactcttgtgggttttggttgtcccttttcattttttaccatgctgtattactagattatactttgataacagagtttaacttttgctacttctcaagatcagttcaaccttgagatttacctgcccatctttttgttgatcttatagtctttgtaattctgcatatggcaaatcttgtcctaaataatactttttttccggatccggccataagggcctgatccgtcgagggggggcccggtaccagcttttgttccctttagtgagggttaatttcgagcttggcgtaatcatggtcatagctgtttcctgtgtgaaattgttatccgct

SEQ ID NO: 7 - DNA, 18 bp, Artificial Sequence
  Description of Artificial Sequence synthetic DNA
tgtaaaacgacggccagt

SEQ ID NO: 8 - DNA, 24 bp, Artificial Sequence
  Description of Artificial Sequence synthetic DNA
agcggataacaatttcacacagga

SEQ ID NO: 9 - DNA, 503 bp, Homo sapiens
ccaggggctgctgctgttgctgctgctgagcatgggcggggcatgggcatccagggagccgcttcggccatggtgccaccccatcaatgccatcctggctgtcgagaaggagggctgcccagtgtgcatcaccgtcaacaccaccatctgtgccggctactgccccaccatgatgcgcgtgctgcaggcggtcctgccgcccctgcctcaggtggtgtgcacctaccgtgatgtgcgcttcgagtccatccggctccctggctgcccgcgtggtgtggaccccgtggtctccttccctgtggctctcagctgtcgctgtggaccctgccgccgcagcacctctgactgtgggggtcccaaagaccaccccttgacctgtgaccacccccaactctcaggcctcctcttcctctaaagaccctccccgcagccttccaagtccatcccgactcctggagccctgacaccccgatcctcccacaataaaggcttctcaatccgca

SEQ ID NO: 10 - DNA, 172 bp, Homo sapiens
ccaggggctgctgctgttgctgctgctgagcatgggcggggcatgggcatccagggagccgcttcggccatggtgccaccccatcaatgccatcctggctgtcgagaaggagggctgcccagtgtgcatcaccgtcaacaccaccatctgtgccggctactgccccaccatg

SEQ ID NO: 11 - DNA, 331 bp, Homo sapiens
atgcgcgtgctgcaggcggtcctgccgcccctgcctcaggtggtgtgcacctaccgtgatgtgcgcttcgagtccatccggctccctggctgcccgcgtggtgtggaccccgtggtctccttccctgtggctctcagctgtcgctgtggaccctgccgccgcagcacctctgactgtgggggtcccaaagaccaccccttgacctgtgaccacccccaactctcaggcctcctcttcctctaaagaccctccccgcagccttccaagtccatcccgactcctggagccctgacaccccgatcctcccacaataaaggcttctcaatccgca

SEQ ID NO: 12 - DNA, 19 bp, Artificial Sequence
  Description of Artificial Sequence synthetic DNA
ccaggggctgctgctgttg

SEQ ID NO: 13 - DNA, 30 bp, Artificial Sequence
  Description of Artificial Sequence synthetic DNA
cagcacgcgcatcatggtggggcagtagcc

SEQ ID NO: 14 - DNA, 30 bp, Artificial Sequence
  Description of Artificial Sequence synthetic DNA
tgccccaccatgatgcgcgtgctgcaggcg

SEQ ID NO: 15 - DNA, 23 bp, Artificial Sequence
  Description of Artificial Sequence synthetic DNA
tgcggattgagaagcctttattg